(19) United States
(12) Patent Application Publication
(10) Pub. No.: US 2025/0017828 A1
(43) Pub. Date: Jan. 16, 2025

(54) PARTICLES CONTAINING COLORING AGENTS AND METHODS OF USING THE SAME

(71) Applicant: Ephemeral Solutions, Inc., Milford, CT (US)

(72) Inventors: Vandan K. SHAH, Milford, CT (US); Brennal PIERRE, Bridgeport, CT (US)

(21) Appl. No.: 18/586,769

(22) Filed: Feb. 26, 2024

Related U.S. Application Data
(63) Continuation of application No. 17/164,308, filed on Feb. 1, 2021, now abandoned.
Application No. 17/164,308 is a continuation of application No. PCT/US2019/046021, filed on Aug. 9, 2019.
(60) Provisional application No. 62/717,584, filed on Aug. 10, 2018.

(51) Int. Cl. A61K 8/11; A61K 8/90; A61Q 1/02

(52) U.S. Cl. CPC A61K 8/11; A61K 8/90; A61Q 1/025; A61K 2800/434; A61K 2800/654; A61K 2800/91

(57) ABSTRACT

The disclosure relates to a composition that is designed to be administered to a subject intradermally for treating pigmentless skin or creating a temporary tattoo. The composition comprises particles having a polymeric shell and a core that includes a coloring agent. The particles are in a carrier solution at a concentration that is cosmetically effective to delay the bioabsorbance and/or biodegradation of coloring agent in a subject's skin. Bioabsorbance and/or biodegradation of the particles fades the tattoo until it is no longer visible.

### CROSS-REFERENCE TO RELATED APPLICATIONS

This application claims priority to U.S. provisional patent application No. 62/717,584, filed Aug. 10, 2018, the entire contents of which are incorporated herein by reference.

### BACKGROUND

Evidence of skin ornamentation dates back to prehistoric times and have been used to signify status (e.g., marital status or military rank), to identify affiliations, and for aesthetic purposes. Tattooing has also been used therapeutically for treating dermatologic conditions, such as hypopigmentation and hyperpigmentation caused by vitiligo, skin grafts, and port-wine stains.

Tattoos are typically applied by depositing ink into the dermis using a tattoo machine (e.g., a tattoo gun). Carriers for the pigment, e.g., water, are absorbed, and the insoluble pigment particles remain in the dermis where initially deposited. The inertness and aggregation results upon deposition of the tattoo ink particles prevent their elimination from the interstitial space of the tissue by the immune system, and therefore leads to its permanent effects.

Over the years, an individual's style, interests, and skin laxity may evolve. Although tattoos may be removed using laser-based methods, such methods are relatively expensive and may not completely eliminate the tattoo. Additionally, surgical removal, dermabrasion, and salabrasion are invasive removal procedures and may lead to scarring. To avoid these drawbacks, some turn to paints that can be drawn on the skin (e.g. henna). These paints, however, are easily washed off and do not provide the receiver with the genuine feeling of having a somewhat permanent tattoo. The desire exists for semi-permanent tattoos that can retain their vibrancy for about 2 months to about 12 months.

### SUMMARY

Various technologies and reagents useful in certain aspects of the device can be readily used by those of ordinary skill in the art with the benefit of the present disclosure. Additional features such as adhesives, coverings such as bandages, syringes which are preloaded for injection intradermally, can be readily incorporated. For example, devices may be injected into a subject, or the device may be administered to or inserted into the skin of a subject.

One aspect of the disclosure relates to a composition comprising a particle and a carrier solution. In one embodiment, the particle that comprises a shell and a core. In one embodiment, the shell comprises a polymer that is bioabsorbable and biodegradable. Exemplary polymers include polycaprolectone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), polyorthoester, aliphatic polyanhydride, and/or aromatic polyanhydrides, or a block copolymer thereof.

In one embodiment, the core comprises a coloring agent having a molecular weight of about 5 to about 10×10⁶ Daltons.

In one embodiment, the carrier solution is a liquid, solid, semi-solid, gel, paste, or wax.

In one embodiment, the particle has a diameter of less than or equal to about 100μm, about 90 μm, about 80 μm, about 70 μm, about 60 μm, about 50 μm, about 40 μm, about 30 μm, about 20 μm, about 15 μm, about 10 μm, about 9 μm, about 8 μm, about 7 μm, about 6 μm, about 5 μm, about 4 μm, about 3 μm, about 2 μm, about 1 μm, or about 0.5 μm. In one embodiment, the particle is sized to induce aggregation upon incorporation into the dermis of an animal or a human.

In one embodiment, the polymer is present in the shell at a concentration effective to induce aggregation upon incorporation into the dermis of an animal or a human. Without wishing to be bound by a particular theory, hydrophobic interactions lead to aggregation of the particles in the physiological milieu. In one embodiment, electrostatic, cross-linking via surface groups, and/or polyelectrolyte interactions give rise to particle aggregation in the dermis of an animal or human. In one embodiment, the polymer is present in the particle in an amount sufficient to prevent or inhibit phagocytosis of the coloring agent.

In one embodiment, the shell has a thickness of about 0.2 μm to 10 μm, about 0.3 μm to 9 μm, about 0.4 μm to 8 μm, about 0.5 μm to 7 μm, about 0.6 μm to 6 μm, about 0.7 μm to 5 μm, about 0.8 μm to 4 μm, about 0.9 μm to 3 μm, about 1 μm to 2 μm, inclusive.

In one embodiment, the polymer has a weight average molecular weight between 50 Da to 100 kDa, inclusive. In one embodiment, the polymer is crystalline, semi-crystalline, or amorphous. In one embodiment, the polymer is cationic, anionic, or zwitterionic at physiological pH. In one embodiment, the polymer undergoes surface or bulk erosion in aqueous solution. In one embodiment, the polymer, the weight average molecular weight, and the shell thickness are configured such that at least one of a bioabsorption profile and a biodegradation profile exhibits a lag phase of about 2 months to about 12 months. After the lag phase, the coloring agent is rapidly released into dermis, absorbed, and/or degraded.

In one embodiment, the shell further comprises a thermoresponsive polymer. In one embodiment, the thermoresponsive polymer induces particle aggregation inducer upon incorporation of the composition into the dermis of an animal or a human. In a preferred embodiment, at a temperature of about 98 degrees Fahrenheit (body temperature) or higher, the particles are aggregated, and, at temperature of less than 98 degrees Fahrenheit, the particles are in a non-aggregated form. In some embodiments, the non-aggregated form of the particles facilitates administration and dispersion of the particles in a subject. In some embodiments, administration of the composition is accomplished by intradermal injection. In one embodiment, the thermoresponsive polymer is Pluronic® F-127. At concentrations of 18-50%, Pluronic® F-127 forms gels above 10° C. It re-liquefies when cooled to below 10° C. In some embodiments, the thermoresponsive polymer is Poly(N-isopropylacrylamide) (PNIPAM), which can be present in the shell in an range of about 0.1% to about 50%, about 0.2% to about 50%, about 0.3% to about 50%, about 0.4% to about 50%, about 0.5% to about 50%, about 1% to about 50%, about 2% to about 50%, about 0.1% to about 50%, about 3% to about 50%, about 4% to about 50%, about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 0.1% to about 49%, about 0.1% to about 48%, about 0.1% to about 47%, about 0.1% to about 46%, about 0.1% to about 45%, about 0.1% to about 40%, about 0.1% to about 35%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, or about 0.1% to about 1% w/w (PNIPAM/particle weight).

In one embodiment, the coloring agent is a dye or a pigment. In one embodiment, the coloring agent is fluorescent or phosphorescent. In one embodiment, the coloring agent is present in the core in an amount between 1 ng and 1 μg, inclusive. In some embodiments, the composition comprises a coloring agent chosen from one or a combination of the following non-limiting examples: melanin, [Phthalocyaninato(2-)] copper, FD&C Red 40 (Food Red 17, Allura Red), FD&C Yellow 5, Nigrosin, Reactive Black 5, Acid Blue 113, Brilliant black BN Granular (Food Black 1), D&C Yellow 10, FD&C Blue 1 (Food Blue 2), FD&C Blue 2, Acid Black 1, Acid Black 24, Acid Black 172, Acid Black 194, Acid Black 210, Spirulina Extract Powder, Gardenia Yellow 98%, Gardenia Yellow 40%, Gardenia Black, Gardenia Blue, Gardenia Red, Cochineal/Carmine, Annatto, Beta carotene, D&C Orange 4, D&C Red 33, D&C Red 22, Ext D&C Violet 2, D&C Yellow 8, FD&C Green 3, FD&C Red 4, FD&C Yellow 6, FD&C Red 3, Ponceau 4R, Acid Red 52, Carmoisine, Amarnath, Brown HT, Black PN, Green S. Patent Blue V, Tartrazine, Sunset Yellow, Quinolline Yellow, Erythrosine, Brilliant Blue, Indigo Carmine, D&C Green 5, D&C Red 17, D&C Red 21, D&C Red 27, D&C Yellow 11, D&C Violet 2, D&C Green 6, D&C Red 30, D&C Red 31, D&C Red 28, D&C Red 7, D&C Red 6, D&C Red 34, D&C Yellow 10, Lake of Carmoisine, Lake of Ponceau 4R, Fanchon Yellow, Toluidine Red, Lake of Acid red 52, Lake of Allura Red, Lake of Tartrazine, Lake of Sunset Yellow, Lake of Brilliant Blue, Lake of Erythrosine, Lake of Quinoline, Lake of Indigo Carmine, Lake Patent Blue V, Lake Black PN, Lithol Rubin B, Iron Oxide Red, Iron Oxide Yellow, Iron Oxide Black, Iron Blue, Titanium Dioxide, D&C Red 36, Carbon Black, Ultramarine Blue, Ultramarine Violet, Ultramarine Red/Pink, Chromium Oxide Green, Mica, Chromium Hydroxide Green, Talc, Manganese Violet, Iron Oxide Burgundy, Iron Oxide Sienna, Iron Oxide Tan, Iron Oxide Amber, Iron Oxide Brown-G, Iron Oxide Brown S, Sodium Copper Chlorophyllin, Caramel, Riboflavin, Canthaxanthin, Paprika, D&C Green 8, Ext D&C Yellow 7, NOIR Brilliant BN, Ferric Ammonium Ferrocyanide, D&C Yellow 10 Lake, FD&C Yellow 5 Lake, FD&C Yellow 6 Lake, D&C Red 21 Lake, D&C Red 33 Lake, FD&C Red 40 Lake, D&C Red 27 Lake, D&C Red 28 Lake, FD&C Blue 1 Lake, D&C Red 30 Lake, D&C Red 36 Lake, D&C Red 6 Lake, D&C Red 7 Lake, D&C Black 2. Combinations of coloring agents are contemplated by the disclosure in such concentrations that are cosmetically effective, such that release into dermis or breaks down in a lag phase in about 2 months to about 12 months. Release and degradation of the contents of each particle layer may result in a partial or full color change of the tattooed design.

In one embodiment, the core consists of the coloring agent, and the coloring agent is an aggregate. In one embodiment, the particle has a diameter of less than or equal to about 10 μm, about 9 μm, about 8 μm, about 7 μm, about 6 μm, about 5 μm, about 4 μm, about 3 μm, about 2 μm, about 1 μm, or about 0.5 μm. In one embodiment, the coloring agent is dissolved or suspended throughout the particle, which need not have a core-shell structure.

In one embodiment, the core further comprises a core polymer. In one embodiment, the polymer and the core polymer are the same or different. In one embodiment, at least one of the polymer and the core polymer is the block copolymer. In one embodiment, the block copolymer comprises a diblock copolymer or a triblock copolymer. In one embodiment, the core polymer is present in the particle at a concentration of about 7%-10%, about 10%-15%, about 15%-20%, about 20%-25%, about 25%-30%, about 30%-35%, about 35%-40%, about 40%-45%, about 45%-50%, about 50%-55%, about 55%-60%, about 60%-65%, about 65%-70%, about 70%-75%, about 75%-80%, about 80%-85%, about 85%-90%, or about 90%-92% w/w.

In one embodiment, the coloring agent is adsorbed to, physically entrapped by, or covalently bonded to the core polymer. Without wishing to be bound, the inventors hypothesize that as the core polymer degrades, the coloring agent releases into dermis with the degraded polymer components and both are removed by the body. In one embodiment, the coloring agent comprises a metal that forms a co-ordinate bond with the core polymer. In one embodiment, the coloring agent is at a concentration of about 0.01% to 10% w/w, 0.02% to 9%, 0.03% to 8%, 0.04% to 7%, 0.05% to 6%, 0.06% to 5%, 0.07% to 4%, 0.08% to 3%. 0.09% to 2%, 0.1% to 1% inclusive, based on a total polymer weight of the particle.

In one embodiment, the core comprises the hydrogel. In one embodiment, the coloring agent is adsorbed to, physically entrapped by, intercalated, non-covalently, or covalently bound with the core polymer covalently bonded to the hydrogel. In one embodiment, the hydrogel comprises at least one of: alginate, chitosan hydrochloride, methacrylate modified hyaluronic acid (HA-MA), thiolated hyaluronic acid (HA-SH), poly(N-isopropylacrylamide) (PNIPAM), and polyethylene glycol (PEG). In one embodiment, the hydrogel comprises a salt of such hydrogels. In some embodiments, the coloring agent comprises a metal that forms a co-ordinate bond with the hydrogel.

In one embodiment, the core further comprises at least one of the following: alginate, pectin, chitosan, hyaluronic acid, k-carrageenan, agarose, agar, cellulose derivatives, carboxy methyl cellulose (CMC), protein-based hydrophilic polymers, collagen hydrolysate, gelatin, synthetic hydrophilic polymers, polyacrylamide, polyacrylic acid, polyvinyl alcohol, polyethylene glycol (PEG) and modified PEG. In one embodiment, the shell or the core further comprises at least one polyanhydrides selected from the group consisting of: poly[bis(p-carboxyphenoxy)methane)] (poly(CPM)), poly[1,3-bis(p-carboxyphenoxy)propane)] poly(CPP), poly[1,6-bis(p-carboxyphenoxy)hexane] (poly(CPH)), poly(sebacic anhydride) (poly(SA)), Poly[1,4-bis(hydroxyethyl)terephthalate-alt-ethyloxyphosphate], and Poly[1,4-bis(hydroxyethyl)terephthalate-alt-ethyloxyphosphate]-co-1,4-bis(hydroxyethyl)terephthalate-co-terephthalate (P(BHET-EOP/BHET), 80/20). In one embodiment, the shell or the core further comprises at least one polyorthoester (POE) selected from the group consisting of: POE I, POE II, POE III, and POE IV, POE I, POE II, POE III, and POE IV are 1^{st}, 2^{nd}, 3^{rd} and 4^{th} generation polyorthoesters, respectively. In one embodiment, the polyorthoesters include a heterocyclic ring.

In one embodiment, the particles are present in the carrier solution at a concentration of about 5 to about 20, about 20 to about 50, about 50 to about 80, about 80 to about 110, about 110 to about 140, about 140 to about 170, about 170 to about 200, about 200 to about 230, about 230 to about 250, about 250 to about 280, about 280 to about 310, about 310 to about 340, about 340 to about 370, about or 370 to about 400 mg/ml. The concentration of particles can also be expressed as a % w/v, wherein

$$\% w / v = \frac{grams of particles}{ml composition} \times 100 \% .$$

In one embodiment, the particles are present in the carrier solution at a concentration of about 5 to about 8, about 8 to about 11, about 11 to about 14, about 14 to about 17, about 17 to about 20, about 20 to about 23, about 23 to about 25, about 25 to about 28, about 28 to about 31, about 31 to about 34, about 34 to about 37, about 37 to about 40, about 37 to about 40, about 40 to about 43, about 43 to about 45, about 45 to about 48, about 48 to about 50, about 50 to about 53, about 53 to about 55, about 55 to about 58, or about 58 to about 60% w/v. In one embodiment, the composition is at a concentration sufficient to maintain osmotic pressure within the particle for at least about 2 months to about 60 months.

In one embodiment, the composition further comprises a humectant, a biocide, a buffer, a surfactant, and/or a copolymer.

In one aspect of the disclosure, a method of tattooing a subject comprises a step of administering to the subject compositions as disclosed in the present application. In one embodiment, the administering step comprises intradermal administration of a cosmetically effective amount of a composition as disclosed herein.

In one embodiment, a method of inhibiting absorption of a coloring agent within the skin of a subject comprises a step of encapsulating the coloring agent into any particle disclosed herein.

Another aspect of the disclosure relates to a method of treating a pigment disorder in a subject in need thereof comprises a step of contacting a portion of the skin of the subject with dysfunctional pigment secretion with a therapeutically effective dose of the particles of any of claims 1 through 44.

Particles of the disclosure are particularly useful for administration of an active medical agent. The compositions may be particularly useful for pediatric, elderly patients, and/or those who suffer from mental illness, who are difficult to test and who are non-compliant, as well as for the military, and people without health insurance (e.g., lower income persons and/or homeless persons).

In one set of embodiments, the method includes an act of altering coloration of an embedded colorant in a subject by administering an electrical, magnetic, and/or a mechanical force to the subject. The method in still another set of embodiments includes an act of determining an analyte in a subject by determining, in the subject, particles having at least two distinct regions, each region being present on the surface of the particles.

Methods according to yet another set of embodiments includes acts of providing a first particle having at least two distinct regions, each region being present on the surface of the first particle, the first particle containing a first coloring agent; providing a second particle (which in some embodiments may have at least two distinct regions, each region being present on the surface of the second particle), the second particle containing a second coloring agent; and causing the first particle and the second particle to become immobilized relative to each other such that the first coloring agent and the second coloring agent are able to react.

Still another embodiment is generally directed to a device for delivery of a plurality of particles to the dermis or epidermis of a subject. According to one set of embodiments, the device contains a substrate; and a plurality of epidermis and/or dermis insertion objects (herein “skin insertion objects), removably connected to the substrate, optionally carrying a coloring agent. In some cases, the substrate is constructed and arranged to apply the plurality of epidermis and/or dermis insertion objects to the skin of a subject and to facilitate introduction of the objects into the epidermis and/or dermis, and is fastened to the plurality of objects at a degree of adhesion such that, when the objects are delivered to the dermis and/or epidermis, at least a portion of the majority of them remain in the dermis and/or epidermis when the substrate is removed from the skin.

Still another aspect is generally directed to a kit for the delivery of a coloring agent to the dermis and/or epidermis. The kit, according to one set of embodiments, includes a plurality of skin insertion objects, at least some of which carry a particulate composition comprising a coloring agent, constructed and arranged such that, when the plurality of skin insertion objects are applied to the skin, at least some of the particulate composition is delivered to and remains in the dermis and/or epidermis for a cosmetically acceptable amount of time.

Without wishing to be bound by a specific theory, the inventors hypothesize that following injection of the ink particles onto a region of skin, the ink particles reside in the interstitial space between dermal cells where they form large aggregates. Additionally, tattoo ink particles invoke a foreign-body inflammatory reaction that is composed of epithelioid cells, lymphocytes, and giant cells that attempt to engulf and internalize the foreign tattoo ink particles and ink particle aggregates. Macrophages and dendritic cells become enlarged and develop into epithelioid cells and multinucleated giant cells. This type of reaction, the size of the ink particle aggregates, and the collagen network surrounding the aggregates are largely responsible for maintaining tattoo ink in the dermis over longer period. As such, after administering the tattoo ink into the dermis, aggregation propensity of particles is crucial for maintaining stability of tattoos during a lag phase in which the shell is expected to bioasorb and/or biodegrade. Smaller particles have higher aggregation propensity due to their larger surface area. Therefore an appropriate particle size range is necessary for ensuring aggregation and achieving good tattoo vibrancy over time. In some embodiments, the particle size is no more than about 100 microns in diameter.

### BRIEF DESCRIPTION OF DRAWINGS

FIG. 1 shows a schematic representation of a particle.

### DETAILED DESCRIPTION OF EMBODIMENTS

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, compositions, polymers, particles, and reagents described, as these may vary. It also is to be understood that the terminology used herein is for the purpose of describing particular embodiments only, and is not intended to limit the scope of the present embodiments or claims.

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

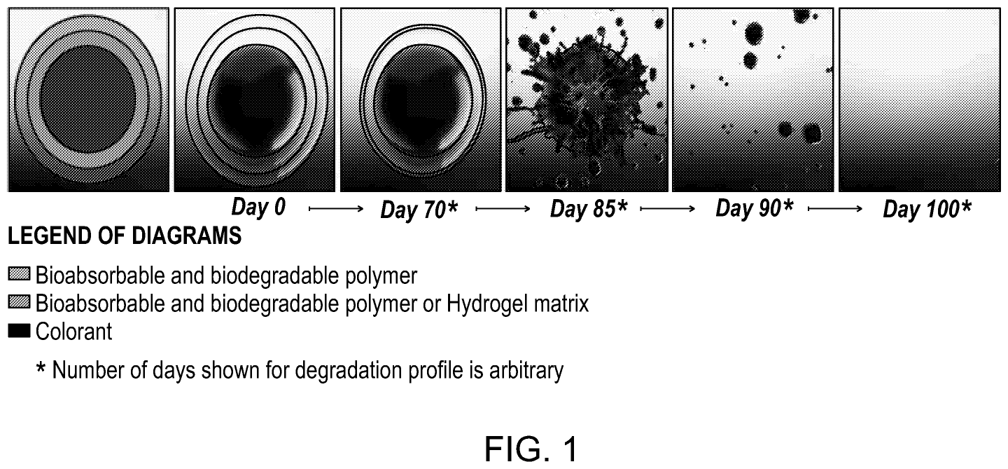

FIG. 1 shows a graphical representation of the bioabsportion and/or biodegradation of one embodiment of a particle of the disclosure over 100 days. FIG. 1A shows a particle that has a core comprising a coloring agent, an inner shell comprising a bioabsorbable and/or biodegradable polymer or hydrogel, and an outer shell comprising a bioabsorbable and/or biodegradable polymer. FIG. 1B is an illustration of one embodiment of a particle at day 0, the day the particle is injected into the skin of an animal or a human. By day 70, the thickness of the outer shell has decreased due to bioabsorption and/or biodegradation as shown in FIG. 1C. This 70-day period is the lag phase during which the coloring agent remains substantially encapsulated by the inner and outer shells, and the tattoo color appears bright under animal or human skin. At about day 85, both the inner and outer shells have degraded sufficiently to allow release of the coloring agent, as shown in FIG. 1D. FIG. 1E shows dispersion, absorption, and/or degradation of the coloring agent, and the tattoo gradually fades. By day 100, the coloring agent and tattoo are no longer apparent (FIG. 1F).

In one embodiment, a composition is provided, wherein the composition comprises: (i) a particle, wherein the particle comprises: (a) a shell comprising bioabsorbable and biodegradable polymer; and (b) a core comprising either similar or different bioabsorbable and biodegradable polymer than the shell or a hydrogel matrix and a coloring agent having a molecular weight between about 5 and about 10×10⁶ Daltons, inclusive; wherein said coloring agent is intercalated, non-covalently, or covalently bound with the polymer or hydrogel matrix; and wherein the bioabsorbable and biodegradable polymer comprises a homopolymer, a copolymer, a block copolymer having two, three, or more blocks (e.g., a diblock or triblock copolymer) chosen from one or a combination of: polycaprolectone (PCL), poly L-lactic acid (PLLA), poly(lactic-co-glycolic acid) (PLGA), polyethylene glycol (PEG), polyethylene glycol-diacrylate (PEGDA), polyorthoester, aliphatic polyanhydride, or aromatic polyanhydride; and (ii) a carrier solution.

Another embodiment provides a composition, wherein the composition comprises: (i) a particle, wherein the particle comprises: (a) a shell comprising bioabsorbable and biodegradable polymer; and (b) a core comprising a coloring agent having a molecular weight between about 5 and about 10×10⁶ Daltons, inclusive; wherein said coloring agent is encapsulated by the shell polymer wherein the shell bioabsorbable and biodegradable polymer comprises a first block or diblock polymer chosen from one or a combination of: polycaprolectone (PCL), poly L-lactic acid (PLLA), poly(lactic-co-glycolic acid) (PLGA), polyethylene glycol (PEG), polyethylene glycol-diacrylate (PEGDA), polyorthoester, aliphatic polyanhydride, poly(sebacic anhydride) (poly(SA)), or aromatic polyanhydride; and (ii) a carrier solution.

As used herein, “particles” are minute portions of matter. The particles may be microparticles and/or nanoparticles. A “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer). In some cases, a plurality of particles may be used, and in some cases, some, or substantially all, of the particles may be the same. For example, at least about 5%, at least about 10%, at least about 20%, at least about 30%, at least about 40%, at least about 50%, at least about 60%, at least about 70%, at least about 80%, at least about 90%, at least about 95%, or at least about 99% of the particles may have the same shape, and/or may have the same or heterogeneous composition.

The particles may be formed of any suitable material, depending on the application. For example, the particles may comprise a glass, and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the particles may comprise a ceramic such as tricalcium phosphate, hydroxyapatite, fluorapatite, aluminum oxide, or zirconium oxide. In some cases (for example, in certain biological applications), the particles may be formed from biocompatible and/or biodegradable polymers such as polylactic and/or polyglycolic acids, polyanhydride, polycaprolactone, polyorthoester, polyethylene oxide, polybutylene terephthalate, starch, cellulose, chitosan, and/or combinations of these. In one set of embodiments, the particles may comprise a hydrogel, such as agarose, collagen, or fibrin.

The particles may include a magnetically susceptible material in some cases, e.g., a material displaying paramagnetism or ferromagnetism. For instance, the particles may include iron, iron oxide, magnetite, hematite, or some other compound containing iron. In another embodiment, the particles can include a conductive material (e.g., a metal such as titanium, copper, platinum, silver, gold, tantalum, palladium, rhodium, etc.), or a semiconductive material (e.g., silicon, germanium, CdSe, CdS, etc.). Other particles include ZnS, ZnO, TiO₂, AgI, AgBr, HgI₂, PbS, PbSe, ZnTe, CdTe, In₂S₃, In₂Sc₃, Cd₃P₂, Cd₃As₂, InAs, or GaAs.

The particles may include other species as well, such as cells, biochemical species such as nucleic acids (e.g., RNA, DNA, PNA, etc.), proteins, peptides, enzymes, nanoparticles, quantum dots, fragrances, indicators, dyes, fluorescent species, chemicals, small molecules (e.g., having a molecular weight of less than about 1 kDa). In some embodiments, in addition to containing one or more reactive agents and/or one or more signaling agents, the particles also contains one or more coloring agents.

In some embodiments, the particles comprise one or a plurality of coloring agents. As used herein, a “coloring agent” is a dye, pigment, or any chemical compound that emits a wavelength of light in the visible spectrum when exposed to visible or ultraviolet light. In some embodiments, the coloring agent is a dye. As used herein, a “dye” refers to a colored molecule that is a liquid or is soluble in a liquid vehicle. In some embodiments, the coloring agent is a pigment. As used herein, a “pigment” refers to a colored molecule that is insoluble in a liquid vehicle. In some embodiments, the coloring agent is one or more fluorophores. In some embodiments, the coloring agent is a combination of two or three of the aforementioned species.

In one embodiment of the present invention, tattoo inks are provided which remain in the dermis for a predetermined period of time (e.g., 2, 3, 6, 9, months or 1, 2, 5, 10 years, etc.) and then spontaneously disappear. These “semi-permanent” or “temporary” tattoo inks are produced by entrapping, encasing, complexing, incorporating, or encapsulating appropriate pigments or coloring agents (pigments which are readily eliminated when present by themselves in the dermis) into vehicles at cosmetically effective concentrations or amounts that allow the pigments or coloring agents to slowly bioabsorb, bioerode, mix and/or biodegrade over a predetermined period of time. In some embodiments, the pigments or coloring agents biodegrade at a constant rate slowly over about a five-year, four-year, three-year, two-year, one-year or half year period, or can release the pigments over a short period of time once a specific percentage of the vehicle has been absorbed. For example, all of the pigment may be released between the fourth and fifth years or any one month period of time between from about 2 and about 60 months.

In some cases, the “tattoo” or particles contained within the skin may be alterable by the administration of an electrical, magnetic, and/or a mechanical force to the subject. For instance, by applying such forces, the particles may be caused to cluster, which may result in a change in color, as discussed above. Thus, one embodiment of the disclosure is directed to a region in the skin of a subject that can be altered by application of an external stimulus, such as an electrical, magnetic, and/or a mechanical force, and/or a chemical applied to the skin (e.g., a chemical which is a binding partner of a species on the particle). In some embodiments, the region of the skin can be altered without electrical, magnetic, or mechanical force and only by adsorption and/or degradation of the particle.

The tattoo (or other mark) present in the skin may have any function, e.g., as a decorative art, or as an identification system. For instance, a tattoo may be verified by applying a stimulus to the subject (e.g., an electric field, a magnetic field, a mechanical force, a chemical, etc.), and confirming the tattoo by identifying a change in the mark, such as a change in color. The change in the mark may be permanent or temporary. As a specific example, a stimulus may be applied to anisotropic particles containing a first region exhibiting a first color and a second region exhibiting a second color. In the absence of the stimulus, the particles exhibit a blend of the first and second colors; however, under application of the stimulus, only one color may be exhibited as the particles are aligned. This identification of a change in color may be used, for example, artistically, or as an identifying mark. As mentioned, in some cases, such a mark may be permanent or temporary. As another example, the particles may be invisible (e.g., non-aggregated) in the absence of a stimulus, but become visible (e.g., aggregated) when a stimulus is applied. In some cases, the particles change their appearance while the stimulus is applied, but revert to their original appearance once the stimulus is removed; in other cases, however, the particles may be able to retain their altered appearance for some time following removal of the stimulus, and in some cases, the particles permanently retain their altered appearance.

As used herein, “dermis” is the thick layer of living tissue below the epidermis that forms one layer of the skin. The dermis may contain blood capillaries, nerve endings, sweat glands, hair follicles, connective tissue, lymphatic vessels, and other structures. The epidermis is the outermost layer of skin, comprising cells that make and store melanin pigment.

As used herein, “biodegradable” or “bioerodable” means capable of being broken down by natural processes. In some embodiments, the natural processes take place within the body of a subject. Similarly, “bioabsorbable”, as used herein, means capable of being absorbed into living tissue.

Any conventional coloring agents suitable for tattoos can be used for the color element of tattoo inks of the present invention, as well as any biologically tolerated colors. The Food and Drug Administration considers the pigments used in tattooing to be “color additives” subject to the FDA color additive regulations under the Federal Food, Drug and Cosmetic Act. [cf. 21 U.S.C. Sections 321(t) and 379(e)]. In addition, virtually any pigment or colored substance tolerated by the body can be used as an appropriate tattoo ink when incorporated with a vehicle to form a pigment/vehicle complex according to the present invention. Non-limiting examples of coloring agents used in the present invention include: melanin, [Phthalocyaninato(2-)] copper, FD&C Red 40 (Food Red 17), FD&C Yellow 5, Nigrosin, Reactive Black 5, Acid Blue 113, Brilliant black BN Granular (Food Black 1), D&C Yellow 10, FD&C Blue 1 (Food Blue 2), FD&C Blue 2, Acid Black 1, Acid Black 24, Acid Black 172, Acid Black 194, Acid Black 210, Spirulina Extract Powder, Gardenia Yellow 98%, Gardenia Yellow 40%, Gardenia Black, Gardenia Blue, Gardenia Red, Cochineal/Carmine, Annatto, Beta carotene, D&C Orange 4, D&C Red 33, D&C Red 22, Ext D&C Violet 2, D&C Yellow 8, FD&C Green 3, FD&C Red 4, FD&C Yellow 6, FD&C Red 3, Ponceau 4R, Acid Red 52, Carmoisine, Amarnath, Brown HT, Black PN, Green S, Patent Blue V, Tartrazine, Sunset Yellow, Quinolline Yellow, Erythrosine, Allura Red, Brilliant Blue, Indigo Carmine, D&C Green 5, D&C Red 17, D&C Red 21, D&C Red 27, D&C Yellow 11, D&C Violet 2, D&C Green 6, D&C Red 30, D&C Red 31, D&C Red 28, D&C Red 7, D&C Red 6, D&C Red 34, D&C Yellow 10, Lake of Carmoisine, Lake of Ponceau 4R, Fanchon Yellow, Toluidine Red, Lake of Acid red 52, Lake of Allura Red, Lake of Tartrazine, Lake of Sunset Yellow, Lake of Brilliant Blue, Lake of Erythrosine, Lake of Quinoline, Lake of Indigo Carmine, Lake Patent Blue V, Lake Black PN, Lithol Rubin B, Iron Oxide Red, Iron Oxide Yellow, Iron Oxide Black, Iron Blue, Titanium Dioxide, D&C Red 36, Carbon Black, Ultramarine Blue, Ultramarine Violet, Ultramarine Red/Pink, Chromium Oxide Green, Mica, Chromium Hydroxide Green, Talc, Manganese Violet, Iron Oxide Burgundy, Iron Oxide Sienna, Iron Oxide Tan, Iron Oxide Amber, Iron Oxide Brown-G, Iron Oxide Brown S, Sodium Copper Chlorophyllin, Caramel, Riboflavin, Canthaxanthin, Paprika, D&C Green 8, Ext D&C Yellow 7, NOIR Brilliant BN, Ferric Ammonium Ferrocyanide, D&C Yellow 10 Lake, FD&C Yellow 5 Lake, FD&C Yellow 6 Lake, D&C Red 21 Lake, D&C Red 33 Lake, FD&C Red 40 Lake, D&C Red 27 Lake, D&C Red 28 Lake, FD&C Blue 1 Lake, D&C Red 30 Lake, D&C Red 36 Lake, D&C Red 6 Lake, D&C Red 7 Lake, D&C Black 2.

One example of a particle, which releases the coloring agent continuously over a predetermined period is one in which the coloring agent is incorporated or mixed in throughout the entire substance of a vehicle to form color-carrying particles. When these coloring agent/vehicle complexes are introduced into the dermis (in the form of a tattoo), the tattoo coloring agent and vehicle slowly bioabsorbs, releasing the coloring agent from the dissolving vehicle, eliminating the coloring agent from the dermis. When all of the coloring agent/vehicle have been absorbed, the tattoo is no longer visible.

To release the coloring agent over a short period of time, bioabsorbable microcapsules or microflakes can be used as the vehicle. With microcapsules, coloring agent/vehicle complexes comprise a core of coloring agent surrounded by the vehicle, which maintains its integrity until a certain threshold percentage of the vehicle is dissolved, bioeroded, or bioabsorbed. At this point, the vehicle no longer protects the coloring agent from elimination. The coloring agent is released into the dermis, where it is eliminated over a relatively short period of time.

Alternatively, microflakes made of coloring agent and vehicle, in which the coloring agent is mixed throughout the microflakes, maintain a relatively consistent coloring agent surface area during the process of bioabsorption. Over a predetermined period of time, the visible coloring agent surface dissolves, similar to the melting of a frozen lake or pond.

The vehicle for the coloring agent comprises any biologically tolerated material that retains the coloring agent in the dermis, for whatever time or under whatever conditions are desired. In any of these cases, the vehicle carries a coloring agent which can be administered into the dermis in any pattern or configuration in a manner similar to conventional tattooing. The vehicle is sufficiently transparent or translucent so as to permit the color of the coloring agent to show through and be visible.

Among other materials that can function as tattoo coloring agent vehicles in the present invention are those which the FDA has found acceptable for use as food additives, including succinylated gelatin, arabinogalactan, glutaraldehyde, petroleum wax, and mixtures thereof. Additional materials for use as tattoo coloring agent vehicles, according to the present invention, include poloxanele, poly(acrylic acid co-hypophosphorite) sodium salt, polyacrylamide, alginate/alginic acid, calcium caseinate, calcium polypectate, cellulose acetate phthalate, cellulose acetate trimellitate, chitosan, edible and natural waxes, fatty acids, fatty alcohols, gellan gums, hydroxy cellulose, hydroxy ethyl cellulose, hydroxy methyl cellulose, hydroxy propyl cellulose, hydro propyl ethyl cellulose, hydroxy propyl methyl cellulose phthalate, lipids, mono-, di- and triglycerides, pectins, phospholipids, polyalkyl (C₁₆-C₂₂) acrylate, polyethylene, oxidized polyethylene, polyethyleneimine reacted with 1,2-dichloroethane, polyoxyethylene(600)dioleate, polyoxyethylene(600)monoricinoleate, polyoxyethylene(23)lauryl ether, polyethylene glycol, polyethylene glycol(400)dioleate, polyethylene glycol(400)mono-& di-oleate, polyglycerol esters of fatty acids, polyisobutylene, polyglycerol phthalate ester of coconut oil fatty acids, polymaleic acid and/or its sodium salts, polyoxyethylene glycol(400)mono-& di-oleates, polyoxyethylene (23) lauryl ether, polyoxyethylene(40)monostearate, polyoxyethylene-polyoxypropylene block polymers, polyoxyethylene(20)sorbitan monooleate, polyoxyethylene (20) sorbitan monostearate, polyoxyethylene(2)sorbitan tristearate, polyoxypropylene glycol, polyvinyl acetate, polysorbate 80, polyvinylpolypyrrolidone, polyvinylpyrrolidone, and poly(20vinylpyridine-co-styrene).

As used herein, “modified PEG” is any polyethylene glycol derivative, for example polyethylene glycol in which one or both of the terminal hydroxyl groups has been previously modified. Suitable PEG derivatives include alkoxy PEGs in which a terminal hydroxyl group(s) has been converted into an alkoxy group.

Other materials for forming the tattoo coloring agent vehicles are biologically tolerated, and include, waxes, polyolefins, or paraffins (e.g., Bayberry, spermaceti, Japan, Ross, etc.), triglycerides, phospholipids, fatty acids and esters thereof (e.g., lauric acid, palmitic acid, sorbitan monopalmitate, sorbitan monostearate, etc.), poly(vinyl palmitate), poly(hexadecyl acrylamide), poly(butyl acrylate), poly(hexadecyl acrylate), poly(octadecyl acrylate), poly(dodecene), poly(isobutene), poly(trimethyl glutarate), polyanhydrides, polyorthoesters, polyesters, polystyrene, polyurethane, polypropylene, polymethacrylate, polytetrafluoroethylene, ceramics, or glasses.

The amount of coloring agent used with the vehicle depends upon the desired color and intensity of the coloring agent, as well as the color and texture of the skin to which the coloring agent is to be administered. To form tattooing ink, the tattoo coloring agent/vehicle complexes are formed into microstructures of desired composition and geometry and suspended in a carrier, such as ethanol or water, or any other conventional tattooing ink fluid, in a concentration sufficient to produce the desired coloration of the skin. Alternatively, the tattoo coloring agent/vehicle complexes are in the form of a suspension in a semi-liquid paste, similar to many conventional tattoo inks. The size of the tattoo coloring agent/vehicle complex is selected so that the ink is easily administered into the dermis with conventional tattoo ink devices.

For producing semi-permanent tattoos, the coloring agents are entrapped, encased, complexed, incorporated, encapsulated, or otherwise associated in or with vehicles composed of bioabsorbable, bioerodable, or biodegradable material. The material is designed to bioabsorb, bioerode, or biodegrade over a predetermined period of time so that the tattoo ink, when administered into the dermis, creates a tattoo which lasts only until the tattoo coloring agent vehicle bioabsorbs. Upon partial or complete bioabsorption of the tattoo coloring agent vehicle, the coloring agent is released, allowing its elimination from the dermis.

A great many biodegradable polymers exist, and the length of time which the tattoo lasts in a visible state in the dermis is determined by controlling the type of material and composition of the vehicle. Among the bioabsorbable, bioerodable, or biodegradable polymers which can be used are those disclosed in Higuchi et al., U.S. Pat. Nos. 3,981,303, 3,986,510, and 3,995,635, including zinc alginate poly(lactic acid), poly(vinyl alcohol), polyanhydrides, and poly(glycolic acid). Alternatively, microporous polymers are suitable, including those disclosed in Wong, U.S. Pat. No. 4,853,224, such as polyesters and polyethers, and Kaufman, U.S. Pat. Nos. 4,765,846 and 4,882,150.

Other polymers which degrade slowly in vivo are disclosed in Davis et al., U.S. Pat. No. 5,384,333, which are biodegradable polymers which are solid at 20-37° C. and are flowable, e.g., a liquid, in the temperature range of 38-52° C. In preparing a semi-permanent tattoo, the coloring agent is incorporated in the polymer matrix, and the system can be warmed to approximately 50° C., where it liquefies. The system is then injected into the dermis in a desired tattoo design, where it cools and resolidifies.

For example, for vehicles which melt, disrupt, weaken, or degrade upon application of heat, a melting temperature of from about 40° C. to about 55° C. is useful. Examples of such heat-labile or meltable materials for fabrication of vehicles include, but are not limited to, those listed in Table 1 or combinations thereof:

TABLE 1
Heat-labile materials

| | Melting |
|---|---|
| | Temperatures |
| Polymer | (° C.) |
| Poly hexadecylester | 43 |
| Poly-n-hexadecyl-acrylamide | 45 |
| Poly butyl ester | 47 |
| Poly-l-dodecene | 45-48 |
| Polyisobutenc | 44-46 |
| Poly(hexadecyl acrylamide) | 45 |
| Poly(butyl acrylate) | 47 |
| Poly(hexadecyl acrylate) | 43 |
| Poly(octadecyl acrylate) | 56 |
| Poly(dodecene) | 45-49 |
| Poly(isobutene) | 44-46 |
| Bayberry wax | 42-48 |
| Spermaceti wax | 42-50 |
| Japan wax | 50-56 |
| Ross wax (refined paraffin wax) | 48-50 |
| Carbowax (polyethylene glycol 1450) | 43-46 |
| Lipoxol 1550 or 2000 (MED PEG-32 or 40) | 40-50 |
| Lauric acid | 44-46 |
| Palmitic acid | 59-61 |
| Sorbitan Monopalmitate | 46-47 |
| Sorbitan Monostearate | 56-58 |
| Softisan (142 or 601 glycerol esters of C₁₀₋₁₈ fatty acids | 40-45 |

For this type of semi-permanent vehicle, any biodegradable polymer system which has the following characteristics can be used, including homopolymers, copolymers, block copolymers, waxes and gels, as well as mixtures thereof. A preferred polymer system is a triblock copolymer of the general formula: [A-B-A]ₓ, where A represents a hydrophobic polymer block, B represents a hydrophilic polymer, and X represents any positive integer from about 1 to about 90,000. The monomers and polymers are preferably linked through ester groups. Preferred hydrophobic polymers and oligomers include, but are not limited to, units selected from polyglycolic acid, polyethylene terephthalate, polybutyl lactone, polycaprolactone, D-polylactic acid, polytetrafluoroethylene, polyolefins, polyethylene oxide, polylactic acid, polyglutamic acid, poly-L-lysine, and poly-L-aspartic acid. Preferred hydrophilic polymers include polyethylene glycol, polypropylene glycol, and poly(vinyl alcohol).

In a preferred embodiment, the particle core comprises the coloring agent and a bioabsorbable and/or biodegradable polymer comprising at least one of polycaprolectone (PCL), poly D-lactic acid (PDLA), poly L-lactic acid (PLLA), poly(lactic-co-glycolic acid), (PLGA), polyethylene glycol (PEG), polyethylene glycol-diacrylate (PEGDA), polyorthoester, aliphatic polyanhydride, and aromatic polyanhydrides, or a block copolymer thereof. The coloring agent can be incorporated into the core polymer by including the coloring agent in the pre-polymer mixture, followed by polymerization. In one aspect of the disclosure, the polymerization process is an emulsion polymerization process. The coloring agent can also be incorporated in the core polymer by dissolving the polymer and the coloring agent in a solvent, followed by evaporation of the solvent. In another aspect of the disclosure, evaporation of the solvent is a single or double emulsion solvent evaporation process. The coloring agent can also be incorporated in the core polymer by melting the core polymer and dissolving and/or suspending the coloring agent directly in the neat polymer melt. It should be appreciated that such methods can be used to incorporate coloring agents into polymers to form layerless particles and/or particle shells.

Hydrogel matrices or vehicles for preparing semi-permanent tattooing inks are formed by cross-linking a polysaccharide or a mucopolysaccharide with a protein and loading the coloring agent into the hydrogel matrices. Proteins include both full-length proteins and polypeptide fragments, which in either case may be native, recombinantly produced, or chemically synthesized. Polysaccharides include both polysaccharides and mucopolysaccharides.

A hydrogel in which the coloring agent can be incorporated to a tattoo ink is disclosed in Feijen, U.S. Pat. No. 5,041,292. This hydrogel comprises a protein, a polysaccharide, and a cross-linking agent providing network linkages there between wherein the weight ratio of polysaccharide to protein in the matrix is in the range of about 10:90 to about 90:10. The coloring agent is mixed into this matrix in an amount sufficient to provide color when the hydrogel matrix is administered to the dermis.

Examples of suitable polysaccharides include heparin, fractionated heparins, heparan, heparan sulfate, chondroitin sulfate, and dextran, including compounds described in U.S. Pat. No. 4,060,081 to Yannas et al. Using heparin or heparin analogs is preferred because there appears to be reduced immunogenicity. The protein component of the hydrogel may be either a full-length protein or a polypeptide fragment. The protein may be in native form, recombinantly produced, or chemically synthesized. The protein composition may also be a mixture of full-length proteins and/or fragments. Typically, the protein is selected from the group consisting of albumin, casein, fibrinogen, gamma-globulin, hemoglobin, ferritin and elastin. The protein component may also be a synthetic polypeptide, such as poly(a-amino acid), polyaspartic acid or polyglutamic acid. Albumin is the preferred protein component of the matrix, as it is an endogenous material which is biodegradable in blood and tissue by proteolytic enzymes. Furthermore, albumin prevents adhesion of thrombocytes and is nontoxic and nonpyrogenic.

In forming hydrogels containing coloring agents, the polysaccharide or mucopolysaccharide and the protein are dissolved in an aqueous medium, followed by addition of an amide bond-forming cross-linking agent. A preferred cross-linking agent for this process is a carbodiimide, preferably the water-soluble diimide, e.g., N-(3-dimethylaminopropyl)-N-ethylcarbodiimide. In this method, the cross-linking agent is added to an aqueous solution of the polysaccharide and protein at an acidic pH and a temperature of about 0° C. to 50° C. preferably from about 4 to about 37° C., and allowed to react for up to about 48 hours. The hydrogel so formed is then isolated, typically by centrifugation, and washed with a suitable solvent to remove uncoupled material.

Alternatively, a mixture of the selected polysaccharide or mucopolysaccharide and protein is treated with a cross-linking agent having at least two aldehyde groups to form Schiff-base bonds between the components. These bonds are then reduced with an appropriate reducing agent to give stable carbon-nitrogen bonds.

Once the hydrogel is formed, it is loaded with the coloring agent by immersing the hydrogel in a solution or dispersion of the coloring agent. The solvent is then evaporated. After equilibration, the loaded hydrogels are dried in vacuo under ambient conditions and stored.

Examples of preferred embodiments of polymers to be used in the preparation of the hydrogel vehicle include one or a combination of alginate, alginate in combination with chitosan hydrochloride, methacrylate modified hyaluronic acid (HA-MA), thiolated hyaluronic acid (HA-SH), poly(N-isopropylacrylamide) (PNIPAM), polyethylene glycol (PEG), polycaprolectone (PCL), poly L-lactic acid (PLLA), poly(lactic-co-glycolic acid) (PLGA), diblock or triblock copolymers in any combination of PCL, PLLA, PLGA or PEG, polyethylene glycol-diacrylate (PEGDA), polyorthoester, and/or aliphatic or aromatic polyanhydrides or aliphatic-aromatic homopolyanhydrides, such as poly[bis(p-carboxyphenoxy)methane)] (poly(CPM)), poly[1,3-bis(p-carboxyphenoxy)propane)] (poly(CPP)), poly[1,6-bis(p-carboxyphenoxy)hexane] (poly(CPH)), poly(sebacic anhydride) (poly(SA)), poly[1,4-bis(hydroxyethyl)terephthalate-alt-ethyloxyphosphate], and/or poly[1,4-bis(hydroxyethyl)terephthalate-alt-ethyloxyphosphate]-co-1,4-bis(hydroxyethyl)terephthalate-co-terephthalate (P(BHET-EOP/BHET), 80/20).

Virtually any coloring agent may be loaded into the hydrogel vehicles, providing that surface considerations, such as surface charge, size, geometry and hydrophilicity, are taken into account. For example, incorporation and release of a high molecular weight coloring agent will typically require a hydrogel having a generally lower degree of cross-linking. The release of a charged coloring agent will be strongly influenced by the charge and charge density available in the hydrogel, as well as by the ionic strength of the surrounding media.

The rate of coloring agent release from the vehicles can also be influenced by post-treatment of the hydrogel formulations. For example, heparin concentration at the hydrogel surface can be increased by reaction of the formulated hydrogels with activated heparin (i.e., heparin reacted with carbonyldiimidazole and saccharine) or with heparin containing one aldehyde group per molecule. A high concentration of heparin at the hydrogel surface will form an extra “barrier” for positively charged coloring agents at physiological pH values. Another way of accomplishing the same result is to treat the hydrogels with positively charged macromolecular compounds like protamine sulfate, polylysine, or like polymers. Another way of varying hydrogel permeability is to treat the surfaces with biodegradable block copolymers containing both hydrophilic and hydrophobic blocks. The hydrophilic block can be a positively charged polymer, like polylysine, while the hydrophilic block can be a biodegradable poly(α-amino acid), such as poly(L-alanine), poly(L-leucine), or similar polymers.

Another slow-release system used as a vehicle for coloring agents to form a semi-permanent tattoo is a coloring agent and an enzyme encapsulated within a microcapsule having a core formed of a polymer which is specifically degraded by the enzyme and a rate controlling skin. The integrity of the shell is lost when the core is degraded, causing a sudden release of coloring agent from the capsule. In this type of system, the microcapsule consists of a core made up of a polymer around which there is an ionically-bound skin or shell. The integrity of the skin or shell depends on the structure of the core. An enzyme is encapsulated with the biologically-active substance to be released during manufacture of the core of the microcapsule. The enzyme is selected to degrade the core to a point at which the core can no longer maintain the integrity of the skin, so that the capsule falls apart. An example of such as system consists of an ionically cross-linked polysaccharide, calcium alginate, which is ionically coated with a polycationic skin of poly-L-lysine. The enzyme used to degrade the calcium-alginate coated with poly-L-lysine microcapsules is an alginase from the bacteria Beneckea pelagio or Pseudomonas putida. Enzymes exist that degrade most naturally-occurring polymers. For example, the capsule core may be formed of chitin for degradation with chitinase. Other natural or synthetic polymers may also be used and degraded with the appropriate enzyme, usually a hydrogenase.

A particularly preferred bioabsorbable polymer vehicle is a triblock copolymer of poly caprolactone-polyethylene glycol-poly caprolactone. This polymer contains ester bonds which hydrolyze in a hydrophilic environment. In some embodiments, the biodegradable polymer matrix should comprise from about 30% to about 99% of the particle.

In some embodiments, the core comprises one or a plurality of: alginate, chitosan hydrochloride, methacrylate modified hyaluronic acid (HA-MA), tholated hyaluronic acid (HA-SH), poly(N-isopropylacrylamide) (PNIPAM), and polyethylene glycol (PEG).

In some embodiments, the shell comprises one or a plurality of: polycaprolactone (PCL); poly L-lactic acid (PLLA); poly(lactic-co-glycolic acid) (PLGA); a diblock or triblock copolymer in any combination of PCL, PLLA, PLGA or polyethylene glycol (PEG); polyethylene glycol-diacrylate (PEGDA); polyorthoester (POE); Poly(N-isopropylacrylamide) (PNIPAM); and aliphatic or aromatic polyanhydrides or aliphatic-aromatic homopolyanhydrides, such as poly(bis(p-carboxyphenoxy)methane) (poly(CPM)), poly(1,3-bis(p-carboxyphenoxy)propane) (poly(CPP)), poly(1,6-bis(p-carboxyphenoxy)hexane) (poly(CPH)), poly(sebacic anhydride) (poly(SA)), Poly(1,4-bis(hydroxyethyl)terephthalate-alt-ethyloxyphosphate), or poly(1,4-bis(hydroxyethyl)terephthalate-alt-ethyloxyphosphate)-co-1,4-bis(hydroxyethyl)terephthalate-co-terephthalate (P(BHET-EOP/BHET), 80/20). In some embodiments, the shell comprises one or a plurality of any of the above polymers, wherein the total polymer weight/weight is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the particle. In some embodiments, the shell comprises one or a plurality of any of the above polymers, wherein the total polymer weight/weight is from about 5% to about 15%, from about 10% to about 20%, from about 15% to about 25%, from about 20% to about 30%, from about 25% to about 35%, from about 30% to about 40%, from about 35% to about 45%, from about 40% to about 50%, from about 45% to about 55%, from about 50% to about 60%, from about 55% to about 65%, from about 60% to about 70%, from about 65% to about 75%, from about 70% to about 80%, from about 75% to about 85%, from about 80% to about 90%, from about 85% to about 95%, or from about 90% to about 99% of the particle.

In some embodiments, the shell comprises polycaprolactone (PCL), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises poly L-lactic acid (PLLA), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises poly(lactic-co-glycolic acid) (PLGA), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle. The ratio of lactide:glycolide in shells comprising PLGA can be about 5:95, about 10:90, about 15:85, about 20:80, about 25:75, about 30:70, about 35:65, about 40:60, about 45:55, about 50:50, about 55:45, about 60:40, about 65:35, about 70:30, about 75:25, about 80:20, about 85:15, about 90:10, or about 95:5.

In some embodiments, the shell comprises a diblock or triblock copolymer in any combination of PCL, PLLA, PLGA or polyethylene glycol (PEG), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises polyethylene glycol-diacrylate (PEGDA), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises polyorthoester (POE), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises aliphatic or aromatic polyanhydrides or aliphatic-aromatic homopolyanhydrides, such as poly(bis(p-carboxyphenoxy)methane) (poly(CPM)), poly(1,3-bis(p-carboxyphenoxy)propane) (poly(CPP)), poly(1,6-bis(p-carboxyphenoxy)hexane) (poly(CPH)), poly(sebacic anhydride) (poly(SA)), Poly(1,4-bis(hydroxyethyl)terephthalate-alt-ethyloxyphosphate), or poly(1,4-bis(hydroxyethyl)terephthalate-alt-ethyloxyphosphate)-co-1,4-bis(hydroxyethyl)terephthalate-co-terephthalate (P(BHET-EOP/BHET), 80/20), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises a diblock copolymer in any combination of poly(bis(p-carboxyphenoxy)methane) (poly(CPM)) and poly(sebacic anhydride) (poly(SA)), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises a diblock copolymer in any combination of poly(1,3-bis(p-carboxyphenoxy) propane) (poly(CPP)) and poly(sebacic anhydride) (poly(SA)), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%. from about 40% to about 90%, from about 45% to about 90%, from about 50% to about 90%. from about 55% to about 90%, from about 60% to about 90%, from about 65% to about 90%, from about 70% to about 90%, from about 75% to about 90%, or from about 80% to about 90% of the particle.

In some embodiments, the shell comprises a diblock copolymer in any combination of poly(1,4-bis(p-carboxyphenoxy)butane) (poly(CPB)) and poly(sebacic anhydride) (poly(SA)), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%, from about 55% to about 90%, from about 60% to about 90%, from about 65% to about 90%, from about 70% to about 90%, from about 75% to about 90%, or from about 80% to about 90% of the particle.

In some embodiments, the shell comprises a diblock copolymer in any combination of poly(1,6-bis(p-carboxyphenoxy)hexane) (poly(CPH)) and poly(sebacic anhydride) (poly(SA)), wherein the polymer weight/weight is from about 5% to about 90%, from about 10% to about 90%, from about 15% to about 90%, from about 20% to about 90%, from about 25% to about 90%, from about 30% to about 90%, from about 35% to about 90%, from about 40% to about 90%, from about 45% to about 90%, from about 50% to about 90%. from about 55% to about 90%, from about 60% to about 90%, from about 65% to about 90%, from about 70% to about 90%, from about 75% to about 90%, or from about 80% to about 90% of the particle.

In some embodiments, the shell and/or core further comprise an aggregation agent. In some embodiments, the aggregation agent is an alkyl cyanoacrylate monomer. The alkyl cyanoacrylate monomer can be methyl cyanoacrylate, n-butyl cyanoacrylate, isobutyl cyanoacrylate, n-hexyl cyanoacrylate, 2-hexyl cyanoacrylate, 2-octyl cyanoacrylate, methoxyisopropyl cyanoacrylate, or a combination thereof. The aggregation agent can be present in the shell and/or the core in a ratio of about 0.2% to about 75%, about 0.3% to about 75%, about 0.4% to about 75%, about 0.5% to about 75%, about 0.6% to about 75%, about 1% to about 75%, about 2% to about 75%, about 3% to about 75%, about 4% to about 75%, about 5% to about 75%, about 10% to about 75%, (g/g), about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 0.2% to about 74%, about 0.2% to about 73%, about 0.2% to about 72%, about 0.2% to about 71%, about 0.2% to about 70%, about 0.2% to about 65%, about 0.2% to about 60%, about 0.2% to about 55%, about 0.2% to about 50%, about 0.2% to about 45%, about 0.2% to about 40%, about 0.2% to about 35%, about 0.2% to about 30%, about 0.2% to about 25%, about 0.2% to about 20%, about 0.2% to about 15%, about 0.2% to about 10%, or about 0.2% to about 5% w/w (aggregation agent/core polymer or aggregation agent/shell polymer).

Several mechanisms are involved in the rate and extent of coloring agent release. In the case of very high molecular weight pigments, the rate of release is more depending on the rate of vehicle bioabsorption. With lower molecular weight pigments, the rate of pigment release is more dominated by diffusion. In either case, depending on the vehicle composition selected, ionic exchange can also play a major role in the overall release profile.

In some embodiments, the coloring agent release may exhibit a “lag phase”, in which degradation is very slow or scarcely appreciable, followed by a rapid release of the coloring agent. The particles of the present invention are designed to be absorbed within a time period of from about 2 to about 12 months after administration. In some embodiments, the particles of the present invention are designed to be absorbed within a time period of from about 3 to about 12 months after administration. In some embodiments, the particles of the present invention are designed to be absorbed within a time period of from about 4 to about 12 months after administration. In some embodiments, the particles of the present invention are designed to be absorbed within a time period of from about 5 to about 12 months after administration. In some embodiments, the particles of the present invention are designed to be absorbed within a time period of from about 6 to about 12 months after administration. In some embodiments, the particles of the present invention are designed to be absorbed within a time period of from about 7 to about 12 months after administration. In some embodiments, the particles of the present invention are designed to be absorbed within a time period of from about 8 to about 12 months after administration. In some embodiments, the particles of the present invention are designed to be absorbed within a time period of from about 9 to about 12 months after administration. In some embodiments, the particles of the present invention are designed to be absorbed within a time period of from about 10 to about 12 months after administration.

In some embodiments, the disclosure relates to a composition or pharmaceutical composition comprising a cosmetically effective amount of a composition of any one or combination of polymers disclosed herein such that the composition prevent absorption of one or plurality of coloring agents in a time period of from about 2 months to about 12months.

The tattoo ink can itself be the vehicle. The vehicle can be a colored particle, which can be, optionally, physically or chemically modified to remain in the dermis indefinitely. Alternatively, these vehicles can be designed to spontaneously dissolve or to be bioabsorbed, causing them to disappear after a predetermined time period to form a semi-permanent tattoo. In other embodiments, these vehicles composed of the pigment are such that they are susceptible to a specific externally applied energy source, such as thermal, sonic (ultrasound), light (e.g., laser light, infrared light, or ultraviolet light), electric, magnetic, chemical, enzymatic, mechanical, or any other type of energy or combination of energies. Treatment of the tattooed skin with the appropriate energy source sufficiently alters the tattoo pigment physically or chemically, allowing its elimination and, thus, erasing the tattoo on demand.

The particles may have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30microns, less than about 25 microns, less than about 10 microns, less than about 3 microns, less than about 1 micron, less than about 300 nm, less than about 100 nm, less than about 30nm, or less than about 10 nm. Preferably, the particles are less than about 100 micron.

The particles may be spherical or non-spherical. For example, the particles may be oblong or elongated, or have other shapes such as those disclosed in U.S. patent application Ser. No. 11/851,974, filed Sep. 7, 2007, entitled “Engineering Shape of Polymeric Micro- and Nanoparticles,” by S. Mitragotri, et al., published as U.S. Publication No. 2008/0112886 on May 15, 2008; International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008; U.S. patent application Ser. No. 11/272,194, filed Nov. 10, 2005, entitled “Multi-phasic Nanoparticles,” by J. Lahann, et al., published as U.S. Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Publication No. 2007/0237800 on Oct. 11, 2007, each of which is incorporated herein by reference. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous. Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks, substantially pyramidal, conical or substantially conical or the like.

As used herein, a “cosmetically effective amount”, “cosmetically effective dose”, or “cosmetically acceptable amount” refers to an amount sufficient to prevent or inhibit phagocytosis of the coloring agent in a subject for a predetermined period of time between from about 1 to about 60 or more months. In some embodiments, the desired cosmetic effect is dependent upon the design being tattooed or the degree to which the tattooed design is desired to be temporary. As such, the cosmetic effect can be a decrease in the time period associated with biodegradation, or release of the coloring agent or agents from the particle and/or inhibition (partial or complete) of phagocytosis of the particles upon administration to a subject or elimination from dermis of the subject. The cosmetically effective amount may also be an amount needed to reduce the toxicity or immunological response elicited after administration t the subject. In some embodiments, the immunological response can be determined based on the age, health, size and sex of the subject. In some embodiments, the cosmetically effective amount can also be determined based on monitoring of the subject's response to treatment.

The term “subject” is used throughout the specification to describe an animal to whom treatment with the compositions according to the present invention is provided or administered. For treatment of those conditions which are specific for a specific subject, such as a human being, the term “patient” may be interchangeably used. In some instances in the description of the present invention, the term “subject” will refer to human subjects. In some embodiments, the subject may be a mammal to whom the present invention is provided or administered. In some embodiments, the subject may be a non-mammalian animal to whom the present invention is provided or administered. In some embodiments, the subject is a domesticated mammal such as a canine, equine, feline, porcine, bovine, murine, caprine, ovine, or other domesticated mammal. In some embodiments, the subject is a human. In some embodiments, the subject is a non-human domesticated farm animal for which tagging or labeling of the skin is desired.

The term “pigment disorder” as used herein, refers to disorders involving skin pigment (e.g., melanin). Examples of pigment disorders include, but are not limited to, all forms of albinism, melasma, pigment loss after skin damage, vitiligo, and any dysfunctional pigment secretion by the skin.

As used herein, “administer” or “administering” refers to any method which delivers the compositions used in this invention to the subject in such a manner so as to be cosmetically effective. Preferably, the compositions are administered into the dermis and/or epidermis layer of the skin.

The term “salt” refers to acidic salts formed with inorganic and/or organic acids, as well as basic salts formed with inorganic and/or organic bases. Examples of these acids and bases are well known to those of ordinary skill in the art. Salts according to the present invention may be used in a variety of forms, for example anhydrous or a hydrated crystalline form. In some embodiments, the salts may be those that are physiologically tolerated by a subject. In some embodiments of the invention, the term “salt” refers to one or more of the anhydrous compounds which find use in purgative products according to the present invention. Salts according to the present invention may be found in their anhydrous form or as in hydrated crystalline form (i.e., complexed or crystallized with one or more molecules of water). Suitable purgative salts for use in the present invention include, for example, monobasic, dibasic, and tribasic salts or a mixture of monobasic, dibasic, and tribasic salts. Salts of the active composition components are prepared with relatively nontoxic acids or bases, depending on the particular substituents found on the compounds described herein. When components of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

As used herein, the terms “treat,” “treated,” or “treating” mean both therapeutic treatment and prophylactic or preventative measures wherein the object is to prevent or slow down (lessen) a physiological condition, disorder or disease, or obtain beneficial or desired clinical results. For purposes of this disclosure, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Thus, “treatment of a pigment disorder” or “treating a pigment disorder” means an activity that prevents, alleviates or ameliorates any of the primary phenomena or secondary symptoms associated with lack of a pigment within a portion or region of a subject's skin. In some embodiments, the symptom associated with a lack of pigment is discoloration of the subject's skin which is improved or altered upon administration of the compositions disclosed herein.

As used herein, the term “Poly(N-isopropylacrylamide)” or “PNIPAM” means a polymer made from the monomer and its functionalized derivatives shown in Table 2, and its functionalized derivatives of Formula I.

TABLE 2

| Chain-end group Functionalized | |
|---|---|
| Poly(N-isopropylacrylamide) (PNIPAm) | Formula |
| Poly(N-isopropylacrylamide) | 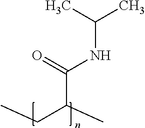 |
| Poly(N-isopropylacrylamide), carboxylic acid terminated | 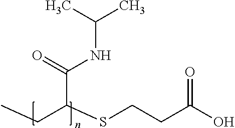 |
| Poly(N-isopropylacrylamide), amine terminated | 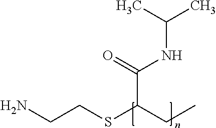 |
| Poly(N-isopropylacrylamide), azide terminated | 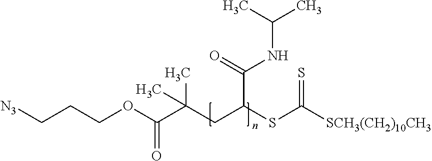 |
| Poly(N-isopropylacrylamide) triethoxysilane terminated | 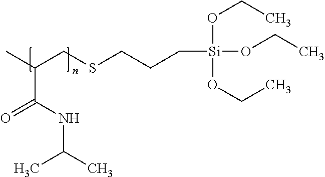 |
| Poly(N-isopropylacrylamide), maleimide terminated | 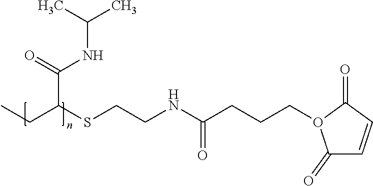 |
| Poly(N-isopropylacrylamide), N-hydroxysuccinimide (NHS) ester terminated | 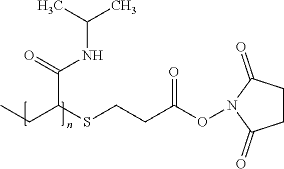 |

N-isopropylacrylamide can be copolymerized with, e.g., methacrylic acid or acrylic acid and a di-acrylamide crosslinker to impart pH and/or temperature sensitivity.

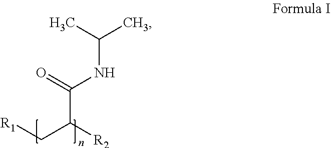

wherein
R₁ is carboxy, hydroxyl, amino, or C₁ to C₃₀ alkyl, alkenyl, alkoxy, phenyl, cycloalkyl, phenoxy, aryl, or alkylamino;
and R₂ is carboxy, hydroxyl, amino, or C₁ to C₃₀ alkyl, alkenyl, alkoxy, phenyl, cycloalkyl, phenoxy, aryl, or alkylamino. In some embodiments, the R₁ and/or R₂ is independently selected as a C₁ to C₂₅, C₁ to C₂₀, C₁, to C₁₅, C₁ to C₁₀, or C₁ to C₅ alkyl, alkenyl, alkoxy, phenyl, cycloalkyl, phenoxy, aryl, or alkylamino.

It is further appreciated that certain features of the disclosure, which are, for clarity, described in the context of separate embodiments, can also be provided in combination in a single embodiment. Conversely, various features of the disclosure which are, for brevity, described in die context of a single embodiment can also be provided separately or in any suitable subcombination.

It is understood that the present disclosure encompasses the use, where applicable, of stereoisomers, diastereomers and optical stereoisomers of any one or plurality of components of the particles described herein, as well as mixtures thereof. Additionally, it is understood that stereoisomers, diastereomers, and optical stereoisomers of the components of the disclosure, and mixtures thereof, are within the scope of the disclosure. By way of non-limiting example, the mixture may include a racemate of coloring agent, polymer, or hydrogel the mixture may comprise unequal proportions of one particular stereoisomer of one or plurality of components in the particle over the others. Additionally, the compounds can be provided as a substantially pure stereoisomers, diastereomers and optical stereoisomers (such as epimers).

The components described herein can be asymmetric (e.g., having one or more stereocenters). All stereoisomers, such as enantiomers and diastereomers, are intended to be included within the scope of the disclosure unless otherwise indicated. Compounds that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C—N double bonds, and the like can also be present in the compounds described herein, and all such stable isomers are contemplated in the present disclosure. Cis and trans geometric isomers of the compounds are also included within the scope of the disclosure and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyitartaric acid, dibenzoyliartane acid, mandelic acid, malic acid, lactic acid, and the various optically active camphorsuifonic acids such as β-camphorsulfonic acid. Other resolving agents suitable for fractional crystallization methods include, but are not limited to. Stereoisomerically pure forms of -methyl-benzyl-arnine (e.g., 5 and R forms, or diastereomerically pure forms), 2-phenylglycinoi, norephedrine, ephedrine, N-methyiephedrine, cyciohexyleihylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Any one or plurality of particle components may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1 H- and 3H-imidazole, 1 H-, 2H- and 4HM,2,4-triazole, 1 11- and 2H-isoindole, and 1 H- and 2H-pyrazole, Tautomeric forms cars be in equilibrium or sterically locked into one form by appropriate substitution.

Particles of the disclosure may include hydrates and solvate forms of any of the components in the particle. For instance, core polymers or hydrogels, matrix material and coloring agents may exist in anhydrous and/or non-solvated forms. Components can also include all isotopes of atoms occurring in the intermediates or final compounds. Isotopes include those atoms having the same atomic number but different mass numbers. For example, isotopes of hydrogen include tritium and deuterium.

In some embodiments, the compounds, or salts thereof, are substantially isolated. Partial separation can include, for example, a composition enriched in the coloring agent or particle of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds or particles and their respective salts are routine in the art.

In some embodiments, the particles may be administered to a subject using a suitable carrier. For example, in one embodiment, the particles are administered via injection. The particles can be administered as solution, suspension, or emulsion. Suitable carriers for injection of the particles include, but are not limited, to sterile saline, phosphate buffered saline, water, ethanol, polyol (for example, glycerol, propylene glycol, and liquid polyethylene glycol, and the like), suitable mixtures thereof, and oil, such as vegetable oils. The formulation may contain one or more pharmaceutically acceptable excipients, such as dispersants, pH modifying agents, buffering agents, surfactants, isotonic agents, preservatives, water soluble polymers (e.g., polyethylene glycols, polyvinyl pyrrolidone, dextran, and carboxymethyl cellulose), temperature responsive polymers (e.g. poly(N-isopropylacrylamide) and their copolymers, poly[2-(dimethylamino)ethyl methacrylate] (pDMAEMA), hydroxypropylcellulose, poly(vinylcaprolactame) and polyvinyl methyl ether) and combinations thereof. Water soluble polymers, temperature responsive polymers (e.g. poly(N-isopropylacrylamide) and their copolymers, poly[2-(dimethylamino)ethyl methacrylate] (pDMAEMA), and hydroxypropylcellulose, poly(vinylcaprolactame) and polyvinyl methyl ether) can be present in the carrier in a range of about 0.1% to about 50%, about 0.2% to about 50%, about 0.3% to about 50%, about 0.4% to about 50%, about 0.5% to about 50%, about 1% to about 50%, about 2% to about 50%, about 0.1% to about 50%, about 3% to about 50%, about 4% to about 50%, about 5% to about 50%. about 10% to about 50%, about 15% to about 50%, about 20% to about 50%, about 25% to about 50%, about 30% to about 50%, about 35% to about 50%, about 40% to about 50%, about 45% to about 50%, about 0.1% to about 49%, about 0.1% to about 48%, about 0.1% to about 47%, about 0.1% to about 46%, about 0.1% to about 45%, about 0.1% to about 40%, about 0.1% to about 35%, about 0.1% to about 30%, about 0.1% to about 25%, about 0.1% to about 20%, about 0.1% to about 15%, about 0.1% to about 10%, about 0.1% to about 5%, about 0.1% to about 4%, about 0.1% to about 3%, about 0.1% to about 2%, or about 0.1% to about 1% w/v of the carrier solution.

In another embodiment, the particles may be administered topically to the surface of a subject's skin or mucosal surface using a suitable carrier. Suitable carriers for topical administration of the particles include gels, foams, ointments, pastes, and lotions. The cream or lotion may contain, for instance, an emulsion of a hydrophobic and a hydrophilic material (e.g., oil and water), distributed in any order (e.g., oil-in-water or water-in-oil), and the particles may be present in any one or more of the emulsion phases.

A “carrier solution”, as used herein, may refer to any of the suitable carriers listed above. In some embodiments, the carrier solution is outside the particle or composition of the present invention. In some embodiments, the carrier solution is within the particle or composition of the present invention. For example, carrier solution may be located between layers of the particle.

“Hydrophilic” as used herein refers to substances that have strongly polar groups that readily interact with water.

“Hydrophobic” as used herein refers to substances that lack an affinity for water; tending to repel and not absorb water as well as not dissolve in or mix with water.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A “lotion” is a low-to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol® homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

As used herein, a “hydrogel” is defined as a substance formed when an organic polymer (natural or synthetic) is set or solidified to create a three-dimensional open-lattice structure that entraps water, or other solution, molecules to form a gel. The solidification can occur, e.g., by aggregation, coagulation, hydrophobic interactions, or cross-linking.

Foams consist of an emulsion in combination with a gaseous propellant. The gaseous propellant consists primarily of hydrofluoroalkanes (HFAs). Suitable propellants include HFAs such as 1,1,1,2-tetrafluorocthane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffer(s) maintain the pH of the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Alternatively, the particles may be mucoadhesive and may be sprayed onto the mucosal surface of the tissue. For example, the particles may be formed from mucoadhesive polymers. Mucoadhesive polymers can be classified in two groups: hydrogels and hydrophilic polymers. Mucoadhesive polymers typically contain functional groups that adhere to tissue, such as carboxylic acid groups, hydroxyl groups, and/or amine groups. Classes of mucoadhesive polymers include, but are not limited to, poly vinylpyrrolidone (PVP), methyl cellulose (MC), sodium carboxy methylcellulose (SCMC) hydroxy propyl cellulose (HPC) and other cellulose derivatives, Carbopol, polyacrylates and crosslinked polyacrylates, chitosan and derivatives thereof (N-trimethyl chitosan), acrylic resins, available under the tradename Eudragits®, poly(dimethyl-aminoethyl methacylate) (PDMAEMA), and combinations thereof.

In some embodiments, the carrier solution comprises a stabilizer. As used herein, a “stabilizer” refers to a substance that when added to a polymeric material, will prevent or slow down the degradation process. See, e.g., Concise Chemical and Technical Dictionary, Fourth Enlarged Edition, Bennet, Chemical Publishing Co., NY, N.Y. (1986).

In some embodiments, the composition further comprises a biocide. As used herein, a “biocide” is any chemical compound that inhibits or prevents pathogen growth. In some embodiments, the biocide is an antibiotic. In some embodiments, the composition further comprises an antimicrobial agent chosen from amikacin, anisomycin, apramycin, azithromycin, blasticidin S, brefeldin A, butirosin, chloramphenicol, chlortetracycline, clindamycin, clotrimazole, cycloheximide, demeclocycine, dibekacin, dihydrostreptomycin, doxycycline, duramycin, emetine, erythromycin, fusidic acid, G438, gentamicin, helvolic acid, hygromycin B, josamycin, kanamycin, kirromycin, lincomycin, meclocycline, mepartricin, midecamycin, minocycline, neomycin, netilmicin, nourseothricin, oleandomycin, oxytetracycline, paromomycin, puromycin, rapamycin, ribostamycin, rifampicin, rifamycin, rosamicin, sisomicin, spectinomycin, spiramycin, streptomycin, tetracycline, thiaphenicol, thiostrepton, tobramycin, tunicamycin, tylosin, viomycin, virginiamycin, camptothecin, 10-deacetylbaccatin III, azacytidine, 7-aminoactinomycin D, 8-quinolinol, 9-dihydro-1,3-acetylbaccatin III, aclarubicin, actinomycin D, actinomycin I, actinomycin V, bafilomycin Al, bleomycin, caprecmycin, chromomycin, cinoxacin, ciprofloxacin, cis-diammineplatinum ii) dichloride, coumermycin Al, L(+)-lactic acid, cytochalasin B, cytochalasin D, dacarbazine, daunorubicin, distamycin A, doxorubicin, echinomycin, enrofloxacin, ctoposide, flumequine, formycin, ganciclovir, metronidazole, mithramycin A, mitomycin C, nalidixic acid, nogalamycin, nonactin, novobiocin, ofloxacin, oxolinic acid, paclitaxel, phenazine, phleomycin, rebeccamycin, sinefungin, streptonigrin, streptozocin, succinylsulfathiazole, sulfadiazine, sulfadimethoxine, sulfaguanidine purum, sulfamethazine, sulfamonomethoxine, sulfanilamide, sulfaquinoxaline, sulfasalazine, sulfathiazole, trimethoprim, tubercidin, 5-azacytidine, formycin A, (+)-6-aminopenicillanic acid, 7-aminodesacetoxycephalosporanic acid, amoxicillin, ampicillin, azlocillin, bacitracin, carbenicillin, cefaclor, cefamandole, cefazolin, cefmetazole, cefoperazone, cefotaxime, cefsulodin, ceftriaxone, cephalexin, cephalosporin C, cephalothin, cephradine, cloxacillin, D-cycloserine, dicloxacillin, D-penicillamine, econazole, ethambutol, lysostaphin, moxalactam, nafcillin, nikkomycin Z, nitrofurantoin, oxacillin, penicillin G, phenethicillin, phenoxymethylpenicillin acid, phosphomycin, pipemidic acid, piperacillin, ristomycin, vancomycin, 2-mercaptopyridine, 4-bromocalcimycin A23187, alamethicin, amphotericin B, calcimycin A23187, chlorhexidine, colistin, hydrocortisone, filipin, gliotoxin, gramicidin A, gramicidin D, ionomycin, lasalocid A, lanomycin, monensin, N-(6-aminohexyl)-5-chloro-1-naphthalenesulfonamide, narasin, nigericin, nisin, nystatin, pimaricin, polymyxin B, DL-penicillamine, polymyxin E, praziquantel, salinomycin, surfactin, valinomycin, (+)-usnic acid, miconazole, 1-deoxymannojirimycin, 2-heptyl-4-hydroxyquinoline-oxide, cordycepin, 1,10-phenanthroline, 6-diazo-5-oxo-L-norleucine, antimycin, antipain, ascomycin, azaserine, bafilomycin, cerulenin, chloroquine, mevastatin, concanamycin A, concanamycin C, cyclosporin A, furazolidone, fusaric acid, geldanamycin, gramicidin C, herbimycin A, indomethacin, lomefloxacin, mycophenolic acid, myxothiazol, netropsin, niclosamide, nikkomycin, methyl-deoxynolirimycin, oligomycin, picricidin A, radicicol, staurosporine, stigmatellin, sulfaguanidine, triacsin C, paraceisin, rifaximin, loracarbef, ertapenem, doripenem, imipenem, cilastatin, meropenem, cefadroxil, cefalotin, cefalothin, cefoxitin, cefprozil, cefuroxime, cefalexin, cefdinir, cefditoren, cefpodoxime, ceftazidime, ceftibulen, ceftizoxime, cefepime, ceftaroline fosamil, ceftobiprole, teicopianin, telavanein, daptomycin, clarithromycin, dirithromycin, roxithromycin, gatifloxacin, levofloxacin, moxifloxacin, norfloxacin, trovailoxacin, grepafloxacin, sparfloxacin, temafloxacin, mafenide, sulfacetamide, silver suladiazine, sulfamethizole, sulfamethoxazole, sulfisoxazole, sulfonam idochrysoidine, clofazimine, dapsone, ethionamide, isoniazid, pyrazinamide, rifabutin, rifapentine, arsphenamine, fosfomycin, mupirocin, platensimycin, quinupristin, dalfopristin, tigecycline, ceftazidime, tinidazoie, artemisinin, artestmate, quinine, sulfadoxine-pyrimethamine, hydroxychloroquine, amodiaquine, pyrimethamine, sulphadoxine, proguanil, mefloquine, atovaquone, primaquine, and halofantrine. In any of the above embodiments, the antimicrobial agent is chosen from gentamicin, imipenem, piperacillin, ceftazidime, aztreonam, ceftriaxone, ampicillin, ciprofloxacin, linezolid, daptomycin, and rifampicirs. In some embodiments, the antimicrobial agent chosen from anisomycin, apramycin, blasticidin S, brefeldin A, butirosin, chlortetracycline, clotrimazole, cycloheximide, demeclocycline, dibekacin, dihydrostreptomycin, duramycin, emetine, fusidic acid, G438, helvolic acid, hygromycin B, kanamycin, kirromycin, lincomycin, meclocycline, mepartricin, midecamycin, netilmicin, nitrofurantoin, nourseothricin, oleandomycin, paromomycin, puromycin, rapamycin, ribostamycin, rifampicin, rifamycin, rosamicin, spectinomycin, spiramycin, streptomycin, thiamphenicol, camptothecin, 10-deacetylbaccatin III, azacytidine, 7-aminoactinomycin D, 8-quinolinol, 9-dihydro-1,3-acetylbaccatin III, aclarubicin, actinomycin D, actinomycin I, actinomycin V, bafilomycin Al, bleomycin, capreomycin, chromomycin, cinoxacin, ciprofloxacin, cis-diammineplatinum (ii) dichloride, coumermycin Al, L(+)-lactic acid, cytochalasin B, cytochalasin D, dacarbazine, daunorubicin, distamycin A, doxorubicin, echinomycin, enrofloxacin, etoposide, flumequine, formycin, fumagillin, ganciclovir, gliotoxin, metronidazole, mithramycin A, mitomycin C, nalidixic acid, netropsin, nitrofurantoin, nogalamycin, nonactin, novobiocin, oxolinic acid, paclitaxel, phenazine, phleomycin, pipemidic acid, rebeccamycin, sinefungin, streptonigrin, streptozocin, succinylsulfathiazole, sulfadiazine, sulfadimethoxine, sulfaguanidine purum, sulfamethazine, sulfamonomethoxine, sulfanilamide, sulfaquinoxaline, sulfasalazine, sulfathiazole, tubercidin, 5-azacytidine, cordycepin, formycin A, (+)-6-aminopenicillanic acid, 7-aminodesacetoxycephalosporanic acid, amoxicillin, ampicillin, azlocillin, bacitracin, carbenicillin, cefaclor, cefamandole, cefazolin, cefmetazole, cefotaxime, cefsulodin, cephalexin, cephalosporin C, cephalothin, cephradine, cloxacillin, D-cycloserine, dicloxacillin, D-penicillamine, econazole, ethambutol, lysostaphin, moxalactam, nafcillin, nikkomycin Z, nitrofurantoin, oxacillin, penicillic, penicillin G, phenethicillin, phenoxymethylpenicillinic acid, phosphomycin, pipemidic acid, piperacillin, ristomycin, vancomycin, 2-mercaptopyridine, 4-bromocalcimycin A23187, alamethicin, amphotericin B, calcimycin A23187, chlorhexidine, clotrimazole, econazole, hydrocortisone, filipin, gliotoxin, gramicidin A, gramicidin C, ionomycin, lasalocid A, lonomycin A, onensin, N-(6-aminohexyl)-5-chloro-1-naphthalenesulfonamide, narasin, nigericin, nisin, nystatin, phenazine, pimaricin, DL-penicillamine, praziquantel, salinomycin, 2-heptyl-4-hydroxyquinolone N-oxide, 1,6-diazo-5-oxo-L-norleucine, 8-quinolinol, antimycin, antipain, ascomycin, azaserine, bafilomycin, cerulenin, chloroquine, cinoxacin, mevastatin, concanamycin A, concanamycin C, coumermycin Al, cyclosporin A, furazolidone, radicicol, rapamycin, staurosporine, sulfaguanidine, triacsin C, trimethoprim, cilastatin, meropenem, cefadroxil, levofloxacin, moxifloxacin, trovafioxacin, grepafolxacin, sparfioxacin, temafloxacin, sulfamethizole, sulfamethoxazole, sulfonamidochrysoidine, clofazimine, dapsone, ethionamide, isoniazid, pyrazinamide, rifabutin, rifapentine, arsphenamine, fosfomycin, mupirocin, platensimycin, quinuprislin, dall pristin, tigecycline, imidazole, artemistin, artesunate, quinine, sulfadoxine-pyrimetbamine, hydroxychloroquinine, amodiaquine, sulphadoxine, proguanil, mefloquine, atovaquone, primaquine, and halofantrine. In some embodiments, the antimicrobial agent is chosen from one or a combination of imipenem, piperacillin, aztreonam, ampicillin, linezolid, daptomycin, and rifampicin.

The amount of the antimicrobial agent can determined based upon known dosage amounts, in some embodiments, the pharmaceutical composition comprises a therapeutically effective amount of the antimicrobial agent. In some embodiments, the amount of antimicrobial agent in the pharmaceutical composition with the arylaniide compound can be reduced by about 10%, by about 20%, by about 30%, by about 40%, by about 50%, by about 60%, by about 70%, by about 80%, or by about 90% compared to administration of the antimicrobial agent by itself.

In some embodiments, the composition further comprises a humectant. As used herein, a “humectant” refers to any substance that promotes retention of moisture. Suitable humectants include polyhydric alcohols or glycerin. Other suitable humectants include polyhydric alcohols such as ethylene glycol, propylene glycol, triethylene glycol, tetraethylene glycol, and sorbitol.

Any particle, carrier solution, or composition disclosed herein may be a component in a pharmaceutical composition. In any such pharmaceutical composition, the composition comprises one or a plurality of disclosed compositions in a pharmaceutically effective amount and one or a plurality of pharmaceutically acceptable carriers. In some embodiments, the pharmaceutical compositions comprise nanoparticles comprising one or a plurality of disclosed compositions in a pharmaceutically effective amount. In some embodiments, the nanoparticles are polymer-containing nanoparticles in homogenous or heterogeneous mixtures, such that, if a mixture is homogenous, the nanoparticles comprise the same or substantially the same compositions disclosed herein. In a heterogeneous mixture, the pharmaceutical composition comprises a plurality of nanoparticles comprising different compositions disclosed herein within each particle or among several particles.

According to the present invention, an improved tattoo ink is provided by incorporating conventional tattoo pigments (e.g., India ink) into vehicles which yield pigment/vehicle complexes that remain in the dermis by virtue of their size, attachment to dermal elements, or encapsulation by cells. In this embodiment of the invention, tattooing inks produce permanent tattoos which have clear lines by entrapping diffusible pigment particles into non-diffusible larger aggregates. Materials used for the vehicle to produce permanent tattoo inks are substances which possess the physical characteristics necessary to remain in the dermis indefinitely. These vehicle materials are used for producing permanent tattoos wherein all of the pigment/vehicle complexes have a sufficiently large size so that the tattoo design does not become blurred by the diffusion of the pigment into adjacent dermis. When tattoo inks contain pigmented particles only of an optimal size, generally from about 10 to about 999 nanometers, there is less blurring of the lines of the tattoo, and the pigment does not partially fade or diffuse into adjacent tissues or become eliminated from the dermis.

Alternatively, the vehicle can bind to dermal elements, such as collagen, elastin, glycosaminoglycans, etc., through ionic, covalent, or other molecular mechanisms. The binding factors include, but are not limited to, natural adhesion molecules, such as fibronectin, laminin, vitronectin, fibrinogen, fibrin, intercellular adhesion molecule-1, and various documented adhesion peptide sequences, such as those containing arginine, glycine, aspartic acid sequences (RGD), other peptide sequences (such as YIGSR), or synthetic adhesives, such as cyanoacrylates.

The term “carrier” includes a pharmaceutical carrier or “excipient”, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular composition form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention. The compositions described herein ears take the form of a solution, suspension, emulsion, tablet, coating of a tablet comprising another active agent, microcapsule, pellet, capsule, capsule containing a liquid, powder, sustained-release formulation, suppository, aerosol, spray, or any other form suitable for topical use. In some embodiments, the compositions disclosed here comprise a gel formulation having one or a plurality of excipients that have no bioactivity and no reaction with the active compound. Excipients of a tablet may include fillers, binders, lubricants and glidants, disintegrators, wetting agents, and release rate modifiers. Binders promote the adhesion of particles of the formulation and are important for a tablet formulation. Examples of binders include, but not limited to, carboxymethylcellulose, cellulose, ethylcellulose, hydroxypropylmethylcellulose, methylcellulose, karaya gum, starch, starch, and tragacanth gum, polyfacrylic acid), and polyvinylpyrrolidone. Topical formulations including 3-methanesuifonylpropionitrile can be in a form of gel, cream, lotion, liquid, emulsion, ointment, spray, solution, suspension, and patches. The inactive ingredients in the topical formulations for example include, but not limited to, lauryl lactate (emollient/permeation enhancer), diethylene glycol monoethylether (emollient/permeation enhancer), DMSO (solubility enhancer), silicone elastomer (rheology/texture modifier), capric triglyceride, (emollient), octisalate, (emollient/UV filter), silicone fluid (emollient/diluent), squalene (emollient), sunflower oil (emollient), and silicone dioxide {thickening agent).

In some embodiments, the pharmaceutically acceptable excipient or carrier is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopocia (EP), the British Pharmacopoeia, and/or the International Pharmacopoeia, which is incorporated herein in its entirety.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Pluronic® F 127, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

One aspect of the disclosure relates to composition or compositions comprising particles either homogenous or heterogeneous species in non-aggregated form at room temperature or from about 65 to about 75 degrees Fahrenheit. In some embodiments, the composition or compositions comprise particles of either homogenous and/or heterogeneous species in non-aggregated form at room temperature or from about 65 to about 75 degrees Fahrenheit, but, when exposed to an analyte at body temperature or from about 98 to about 100 degrees Fahrenheit, the particles aggregate. It should be noted that aggregation and non-aggregation of the particles may not be induced by exposure of particles to an analyte. In another set of embodiments, for example, the clustering or aggregation properties of the particles is externally controlled in some fashion. For instance, an electrical, magnetic, and/or a mechanical force can be used to bring the particles closer together and/or cause the particles to separate. Thus, in some cases, the application of an electrical, magnetic, and/or a mechanical force to the particles causes the particles to exhibit a change in color and/or increase the rate of dispersion upon administration. The clustering or aggregation of particles as discussed herein is not limited to generally spherical aggregations. In some cases, the particles may cluster onto a surface, or the particles may be aligned in some fashion relative to the surface due to an analyte or other external force.

In addition, it should be noted that the particles may contain reaction entities that are not necessarily binding partners to an analyte. For instance, there may be a first layer containing a first reaction entity and a second layer or cavity comprising a second reaction entity that reacts with the first reaction entity; when the particles or contents of cavities are brought together in some fashion (e.g., by exposure to an analyte or other chemical that is recognized by binding partners on each of the particles, by the application of an electrical, magnetic, and/or a mechanical force to bring the particles closer together, or biodegradation, etc.), the first and second reaction entities may react. As a specific example, the reaction between the first and second reaction entities may be an endothermic or an exothermic reaction; thus, when the particles are brought together, a temperature change is produced, which can be determined in some fashion. As another example, a reaction between the first and second reactants may cause the release of a material. In some cases, the material may be one that can be sensed by a subject, e.g., capsaicin, an acid, an allergen, or the like. Thus, the subject may sense the change as a change in temperature, pain, itchiness, swelling, or the like. In some embodiments, the exposure of a first reaction entity with a second reaction entity chemically modifies a coloring agent such that the color of the design may be altered.

In some cases, the particles may be suspended in a carrying fluid, e.g., saline, or the particles may be contained within a matrix, e.g., a porous matrix that is or becomes accessible by interstitial fluid after delivery, or a hydrogel matrix, etc. For instance, the matrix may be formed from a biodegradable and/or biocompatible material such as polylactic acid, polyglycolic acid, poly(lactic-co-glycolic acid), etc., or other similar materials.

In some cases, the matrix may prevent or at least inhibit an immunological response by the subject to the presence of the particles, while allowing equilibration of analytes, etc. with the particles to occur, e.g., if the matrix is porous. For instance, the pores of a porous matrix may be such that immune cells are unable to penetrate, while proteins, small molecules (e.g., glucose, ions, dissolved gases, etc.) can penetrate. The pores may be, for instance, less than about 5 micrometers, less than about 4 micrometers, less than about 3 micrometers, less than about 2 micrometers, less than about 1.5 micrometers, less than about 1.0 micrometers, less than about 0.75 micrometers, less than about 0.6 micrometers, less than about 0.5 micrometers, less than about 0.4 micrometers, less than about 0.3 micrometers, less than about 0.1 micrometers, less than about 0.07 micrometers, and in other embodiments, or less than about 0.05 micrometers. The matrix may comprise, for example, biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these, and/or other materials such as agarose, collagen, fibrin, or the like.

### Methods

Embodiments of the disclosure relate to methods of administering the compositions and pharmaceutical compositions of the disclosure. Particles can be administered by a typical tattooing machine to deliver the particles into the dermis of the subject. The tissue marking procedure traditionally consists of piercing the skin with needles or similar instruments to introduce ink that typically includes inert and insoluble pigment particles having a wide distribution of sizes, which are suspended in a liquid carrier. Examples of machines typically used to apply a tattoo include an electromagnetic coil tattooing machine (such as that disclosed in U.S. Pat. No. 4,159,659 to Nightingale); a rotary permanent cosmetics application machine (such as that disclosed in U.S. Pat. No. 5,472,449 to Chou); or any manual tattooing device (such as the sterile single-use device marketed by Softap Inc., San Leandro, Calif.).

Polymer microspheres encapsulated with dye/pigment can be prepared using a wide variety of methods: solvent-in-emulsion evaporation, phase separation, coacervation, spray drying, crosslinking/gelation, hot melting, grinding, electrospraying, and polymerization (emulsion, suspension, dispersion, and precipitation). For polymerization techniques the starting material is unsaturated monomer molecules, which, upon chain-growth polymerization, will form the beads. For all the other techniques described afterward the starting material is already the polymer.

Emulsions. There are two types of single emulsion techniques: oil-in-water (o/w) and water-in-oil emulsions (w/o). For example, the micro particulate carriers of natural polymers i.e. those of proteins and carbohydrates are prepared by these single emulsion techniques. The natural polymers are dissolved or dispersed in aqueous medium followed by dispersion in non-aqueous medium like oil. In the next step, the cross linking of the dispersed globule is carried out. The cross linking can be achieved either by means of UV light or heat or by using the chemical cross linkers. The chemical cross linking agents used are glutaraldehyde, formaldehyde, acid chloride etc. The nature of the surfactants used to stabilize the emulsion phases can greatly influence the size, size distribution, surface morphology, loading, dye/pigment release, and bio performance of the final multiparticulate product.

Double emulsion method of microspheres preparation involves the formation of the multiple emulsions or the double emulsion of type w/o/w and is best suited for water soluble dyes/pigments. This method can be used with both the natural as well as synthetic polymers. The aqueous dye/pigment solution is dispersed in a lipophilic organic continuous phase. The continuous phase is generally consisted of the polymer solution that eventually encapsulates of the dye/pigment contained in dispersed aqueous phase. The primary emulsion is subjected then to the homogenization or the sonication before addition to the aqueous solution of the poly vinyl alcohol (PVA). This results in the formation of a double emulsion. The emulsion is then subjected to solvent removal either by solvent evaporation or by solvent extraction.

Spray Drying. In Spray Drying technique, the polymer is first dissolved in a suitable volatile organic solvent such as dichloromethane, acetone, etc. The dye/pigment in the solid form is then dispersed in the polymer solution with high-speed homogenization. This dispersion is then atomized in a stream of hot air. The atomization leads to the formation of the small droplets or the fine mist from which the solvent evaporates instantaneously leading the formation of the microspheres in a size range 200 nm-100 μm. The size can be manipulated by modifying several parameters, such as concentration of the polymer, solution flow rate, spraying rate, and drying temperature. Micro particles are separated from the hot air by means of the cyclone separator while the trace of solvent is removed by vacuum drying. One of the major advantages of this process is feasibility of operation under aseptic conditions.

Solvent Evaporation. This process is carried out in a liquid manufacturing vehicle phase. The microcapsule coating is dispersed in a volatile solvent which is immiscible with the liquid manufacturing vehicle phase. A core material (dye/pigment) to be microencapsulated is dissolved or dispersed in the coating polymer solution. With agitation the core material mixture is dispersed in the liquid manufacturing vehicle phase to obtain the appropriate size microcapsule. The mixture is then heated if necessary to evaporate the solvent for the polymer of the core material is disperse in the polymer solution, polymer shrinks around the core. If the core material is dissolved in the coating polymer solution, matrix-type microcapsules are formed. The core materials may be either water soluble or water insoluble materials. Solvent evaporation involves the formation of an emulsion between polymer solution and an immiscible continuous phase whether aqueous (o/w) or non-aqueous.

Phase separation coacervation technique. This process is based on the principle of decreasing the solubility of the polymer in organic phase to affect the formation of polymer rich phase called the coacervates. In this method, the dye/pigment particles are dispersed in a solution of the polymer and an incompatible polymer is added to the system which makes first polymer to phase separate and engulf the dye/pigment particles. Addition of non-solvent results in the solidification of polymer. Poly lactic acid (PLA) microspheres have been prepared by this method by using butadiene as incompatible polymer. The process variables are very important since the rate of achieving the coacervates determines the distribution of the polymer film, the particle size and agglomeration of the formed particles. The agglomeration must be avoided by stirring the suspension using a suitable speed stirrer since as the process of microspheres formation begins the formed polymerize globules start to stick and form the agglomerates. Therefore the process variables are critical as they control the kinetic of the formed particles since there is no defined state of equilibrium attainment.

Solvent extraction. Solvent evaporation method is used for manufacturing of microparticles containing dye/pigment, involves removal of the organic phase by extraction of the non aqueous solvent. This method involves water miscible organic solvents as isopropanol. Organic phase can be removed by extraction with water. This process decreases the hardening time for the microspheres. One variation of the process involves direct incorporation of the dye or pigment to polymer organic solution. Rate of solvent removal by extraction method depends on the temperature of water, ratio of emulsion volume to the water and solubility profile of polymer.

Quasi emulsion solvent diffusion. A novel quasi-emulsion solvent diffusion method to manufacture the controlled release microspheres of drug with acrylic polymers has been reported in the literature. Microparticles can be manufactured by a quasi emulsion solvent diffusion method using an external phase containing distilled water and polyvinyl alcohol. The internal phase consists of dye/pigment, ethanol and polymer. The concentration of polymer is in order to enhance plasticity. At first, the internal phase is manufactured at 60° C. and then added to the external phase at room temperature. After emulsification process, the mixture is continuously stirred for 2 hours. Then the mixture can be filtered to separate the microparticles. The product is then washed and dried by vacuum oven at 40° C. for a day.

Polymerization techniques. The polymerization techniques conventionally used for preparing the microspheres are mainly classified as: I. Normal polymerization II. Interfacial polymerization. Both are carried out in liquid phase.
I. Normal polymerization: It is carried out by using different techniques as bulk, suspension, precipitation, emulsion and micellar polymerization methods. In bulk, a monomer or a combination of monomers along with the initiator or catalyst is usually heated to initiate polymerization. Polymer so obtained may be molded as microspheres. Dye/pigment loading may be done during the polymerization process. Suspension polymerization also referred as bead or pearl polymerization. It is carried out by heating the monomer or composition of monomers as droplets dispersion in a continuous aqueous phase. Droplets may also contain an initiator and other additives. Emulsion polymerization deviates from suspension polymerization as due to the presence initiator in the aqueous phase, which afterwards diffuses to the surface of micelles. Bulk polymerization has merits of formation of pure polymers.
II. Interfacial polymerization: This involves the reaction of various monomers at the interface between the two immiscible liquids to form a film of polymer that essentially envelops the dispersed phase.

PH-triggered microparticle. Microparticles that are designed to release their payload when exposed to acidic conditions are provided as a vehicle for dye/pigment release. Any dye/pigment may be encapsulated in a lipid-protein-sugar or polymer matrix with a PH-triggering agent to form microparticles. Preferably the diameter of the pH triggered microparticles ranges from 50 nm to 10 micrometers. The matrix of the particles may be prepared using any known lipid (e.g., DPPC), protein (e.g., albumin), or sugar (e.g., lactose). The matrix of the particles may also be prepared using any synthetic polymers such as polyesters. The process of formulation include providing an agent & contacting with a PH triggering agent & component selected from lipid, proteins, sugars & spray drying the resultant mixture to create microparticles. Typically, the pH triggering agent is a chemical compound including polymers with a pKa less than 7. The PH triggered microparticles release the encapsulated dye/pigment when exposed to an acidic environment.

Microfluidic. Microfabrication using microfluidic methods has been reported to synthesize monodisperse microparticles. By generating highly monodisperse emulsion of polymer and dye/pigment droplets, easily controlled with the combination of driving pressures of two immiscible fluids and geometry of microchannels, microspheres containing dye/pigment with <5% mean deviation diameters can be obtained at a high throughput.

Crosslinking/gelation. Sol-gel or gelation methods are used for fine-particle production. The gelation method uses a polymeric solution containing dye/pigment, starting from a sol state (colloidal solution) that evolves into a gel state (particles), which is extruded and submerged in a coagulation solution, which acts as a crosslinking agent of the polymer.

Electrohydrodynamic processes or Electrospraying. Electrohydrodynamic processes or Electrospraying is a one-step technique which has potential to generate narrow size distributions of submicrometric particles, with limited agglomeration of particles and high yields. The principles of electrospraying are based on the ability of an electric field to deform the interface of a liquid drop, established by Lord Rayleigh in 1882. The electrospraying process is conceptually simple: a polymer solution is loaded into a syringe and infused at a constant rate using a syringe pump through a small but highly charged capillary (e.g., a 16-26 gauge needle). The applied voltage used is typically up to + or −30 kV and the collector might be placed at a 7 to 30 cm distance from the capillary. Once the droplets have detached from the Taylor cone, the solvent evaporates, generating dense and solid particles, propelled towards the collector. In the context of dye/pigment loading, the dye/pigment is mixed to the polymer solution before electrospraying. Further, the size of the final product can be controlled by manipulating the governing factors such as the system, solution, instrumental and ambient parameters. The system parameters include the molecular weight and the microstructural feature of the polymer. The type and concentration of the polymer and solvent used, determine the solution properties namely pH, conductivity, viscosity and surface tension. The instrumental parameters include electrical potential applied, flow rate of the solution, distance between the tip of the needle and the collector and occasionally the nature of collector material. Additionally, the ambient conditions such as the temperature, humidity and air velocity in the process chamber together determine the rate of evaporation of the solvent from the electrosprayed product.

Hot melting. This method has been also applied in pharmaceutical field to prepare sustained-release tablets and transdermal drug delivery systems. It can also be applied in ink particle preparation. This technique employs polymers with low melting point. The polymers are heated into the molten phase and then dispersed in a suitable dispersion medium containing dye/pigment and slowly cooled and fabricated into microsphere format. Microspheres with a SD between 1% and 5% have been reported.

Precision Particle Fabrication Technology (PPF Technology). Precision particle fabrication (PPF) is a technology developed to produce uniform particles of a variety of materials and adapted for fabrication of controlled-release microparticle systems comprising biodegradable polymers. The main apparatus of PPF is based on passing a fluid containing the sphere-forming material(s) (i.e. biodegradable polymers) and any dye/pigment to be encapsulated through a small (10-100 μm) orifice to form a smooth, cylindrical stream. To break the stream into uniform droplets, the nozzle is acoustically excited by a piezoelectric transducer driven by a wave generator at a defined frequency. By employing an annular flow of a non-solvent phase, called the carrier stream, surrounding the polymer-dye/pigment jet to provide additional “drag” force, microparticle size and shape can be further controlled; particles even smaller than the nozzle openings can be generated.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

### EXAMPLES

### Example 1

### Preparation of PLLA/PLGA Particles With Water-Soluble Coloring Agent

Fabrication of double-walled particles combines the phenomenon of phase separation of two polymers in organic solvent when critical concentrations are attained and the process of solvent evaporation. Coloring agent-loaded particles are prepared by this modified oil-in-oil-in-water (O/O/W) emulsion solvent evaporation technique, utilizing the polymer incompatibility between PLLA and PLGA which results in their complete phase separation.

Separate solutions of PLLA and PLGA in dichloromethane (DCM) (15-20%, w/v) are prepared. Typical DCM volumes used are between 335 and 1000 μl. The preparation of the PLGA polymeric solution slightly differs, in that the coloring agent is added to DCM, sonicated using an ultrasonic probe (model XL2000, Misonix, NY, USA) at 2 W output for 30 seconds to break down any crystals of the coloring agent into smaller filaments (<20 μm), prior to the addition of the polymer PLGA. The two polymeric solutions are then added together and sonicated at 2 W for 20 seconds to create an oil-in-oil (O/O) emulsion, evident with the originally clear polymeric solutions becoming translucent with a milky look. Addition of the emulsion dropwise into 200 ml of nonsolvent of PVA aqueous solution (2.5%, w/v) creates an O/O/W emulsion.

Stirring using a mechanical stirrer at the rate of 250 rpm for 4 hours will allow for the extraction and evaporation DCM as well as the hardening of the particles. Filtration, washing and freeze-drying under vacuum follows. Fabricated particles are stored in a desiccator to prevent hydrolytic degradation of the biodegradable polymer under humidity. Microspheres having varying shell thickness and core diameter are prepared in the same manner by altering the polymer mass ratio (w/w) of PLLA and PLGA ranging from 3:1 to 1:1. Single polymer (PLLA and PLGA) particles intended for characterization and baseline comparison are also prepared using the well-established single emulsion method commonly known in the art.

The morphology of both unloaded and coloring agent-loaded particles are studied with a scanning electron microscope, where the surface and cross-sectional morphology as well as degradation of the particles at various stages of in vitro release were investigated. The particles to be examined are first cross-sectioned using a microtome blade with a frozen holding media and mounted onto metal stubs with double-sided carbon tape. The samples are air-dried before being coated with a layer of platinum using an auto fine coater.

Observations using optical microscope are carried out to identify different polymer layers in the double-walled particles based on the difference in crystalline structures as well as to identify the distribution of the coloring agent within the loaded particles. In preparation for optical microscopy, the microspheres are sectioned using a microtome blade and mounted onto glass slides for viewing under cross Polaroid.

Particle size distributions and mean particle sizes are determined using Coulter laser diffraction particle size analyzer. Particles are suspended in ultrapure water and allowed to flow through the analyzer.

For determining the composition of the core and shell polymer, the differential solubility of the polymer pair PLLA and PLGA in ethyl acetate is utilized. PLGA is soluble but not PLLA. The double-walled particles are first cross-sectioned approximately at the centerline. Each half is then immersed individually into a small amount of ethyl acetate for dissolution for about 10 minutes with little or no agitation. The remnant of the cross-sectioned particle is then removed for optical observation. The solution is also examined to ensure that the core has not fallen out in any case. Hence, two possible scenarios of either a hollow core or the remnant of a core could result depending on whether the core or the shell dissolves. Optical microscopic observations of cross-sectional views will enable the identification of the remaining PLLA polymer as either that of the shell or the core and if they were completely phase separated.

This method is employed together with IR study using Fourier transformed infrared (FTIR) spectra obtained using FTIR microscope connected to FTIR spectrophotometer mainframe and analyzed using Bio-Rad analysis software in the mid IR range (wave number 400-4000 cm⁻¹, resolution 2 cm⁻¹). Standard particles of single polymer and double-walled composite particles are cross-sectioned into halves and mounted on a gold slide for examination. Ten points are randomly selected in the core and shell using the software to obtain the transmission spectra. An average of these spectra are obtained and compared with that of the single polymer particles, used as reference for analysis of the composition of respective zones.

Encapsulation efficiency is defined as the ratio of actual to theoretical loading of the coloring agent within the particles as described in the equation:

$$Efficiency ( \% ) = \frac{{C}_{actual}}{{C}_{theoretical}} \times 100$$

where C_{actual} (mg) is the actual amount of coloring agent contained in particles and C_{theoretical} (mg) the theoretical loading that is equal to total amount of coloring agent used initially. The actual amount of coloring agent encapsulated within the particles is determined using an extraction method where 5 mg of microspheres are accurately weighed out in triplicate and dissolved in 2 ml of DCM, chloroform or dimethyl sulfoxide (DMSO) each.

Extraction of the coloring agent is carried out with the use of 5 ml of deionized water where the water-soluble coloring agent will preferentially partition. The solution with two immiscible phases is then centrifuged at 90.6 g for 10 minutes before the top layer of water is extracted, filtered of any residual particles and analyzed for its coloring agent concentration using high-performance liquid chromatography (HPLC).

For release studies, coloring agent loaded particles (5±0.5 mg) are accurately weighed in triplicates and placed in vials containing 1.8 ml of PBS (pH 7.2). The vials are maintained at physiological temperature of 37° C. in a thermostat oscillating waterbath at 120 rpm. A 1.8 ml volume of the aliquots are collected at preselected times after centrifugation at 90.6 g for 5 minutes and the vials replaced with the same amount of freshly prepared PBS. The coloring agent content in the supernatant is analyzed using HPLC. The peak areas obtained were compared against calibration to determine the coloring agent concentration and the fraction of coloring agent released at each data point calculated. A fresh amount of PBS is added to the particles to replace the removed supernatant.

Irradiations of samples are carried out using a Gamma Chamber (⁶⁰Co. source, half life 5.27 years) with dosage of 50 Gy, 25 kGy applied to the samples at a dose rate of 2.5 Gy/h. Dry ice is added to the sample during the course of radiation to lower the local temperature of the sample and to prevent the sample from undergoing thermal degradation. This is a common practice when high irradiation doses are employed.

Thermal analysis of the particles is performed using a modulated differential scanning calorimeter equipped with controller connected to a cooling system. The samples (about 6.5 mg) are placed in sealed aluminum pans and are subjected to heating from −20° C. to 200° C. for the first heating ramp, cooled to −10° C. and reheated on the second ramp to 200° C. all at a rate of 10° C./min. Data obtained are processed on TA universal analyzer software and glass transition temperatures (T_{g}) and crystalline melting points (Tₘ) identified.

Degradation studies are carried out according to the following procedures: loaded particles and blank particles (20±5 mg) are each accurately weighed and placed in vials containing 10 ml of PBS buffer maintained at 37° C. in a thermostat oscillating waterbath at 120 rpm. The microspheres are removed at predesignated times for extensive study using SEM and DSC. SEM studies are carried out on the loaded microspheres to study the effect of polymer degradation on coloring agent release and the relation between polymer physical properties and characteristics points in the release profile. Blank particles are intended for thermal DSC study to characterize any change in polymer T_{g} and Tₘ under degradation.

### Example 2

### Preparation of POE/PLGA Particles With Water-Soluble and Insoluble Coloring Agents

The coloring agent-loaded double-walled polyorthoester/poly(lactide-co-glycolide) (POE/PLGA) particles with 50% POE in weight are prepared by using a water-in-oil-in-water double emulsion solvent evaporation method. Briefly, 300 mg POE, 300 mg PLGA and 70 mg of water-insoluble coloring agent (CA1) are dissolved in 12 ml DCM (the organic phase); 70 mg water-soluble coloring agent (CA2) is dissolved in 0.15 ml water containing 0.2% (w/v) PVA (the internal aqueous phase). The two solutions are mixed and sonicated for 15 seconds to produce the first water-in-oil emulsion. The emulsion is then poured into 250 ml PBS (pH 7.4) containing 0.2% (w/v) PVA as an emulsifier (the external aqueous phase) to produce a water-in-oil-in-water double emulsion, which is stirred at a constant temperature (15° C.) for 3.5 hours using a mixer controlled by a low temperature circulator. The resultant particles are filtered, washed, freeze-dried overnight and stored at 4° C.

The neat POE and PLGA particles containing CA1 or CA2 are prepared by the same method as detailed above. The internal aqueous phase is still used for the fabrication of the CA1-loaded double-walled POE/PLGA particles.

For the determination of CA2 encapsulation efficiency. 10 mg particles are dissolved in 1 ml DCM and kept at room temperature for about 30 minutes. After dissolution of particles, 10 ml PBS buffer (pH 7.4) is added and the mixture is shaken vigorously for 2 minutes. The mixture is left to stand at room temperature for 1 hour before the aqueous layer is drawn out. The aqueous solution is then filtered. CA2 content in the filtered solution is analyzed using high-performance liquid chromatography (HPLC).

For the determination of CA1 encapsulation efficiency, 5 mg particles are dissolved in 1 ml DCM. After dissolution of particles, 5 ml hexane is added to precipitate polymers and extract CA1. The mixture is filtered and the filtrate is dried. A 20 ml volume of acetonitrile/water (85:15, v/v) is added to dissolve the solid sample. The CA1 content is analyzed by HPLC.

The coloring agent loading and encapsulation efficiency are calculated as the ratio of coloring agent to polymer contents and of actual to theoretical coloring agent contents, respectively.

The surface and internal morphologies of particles before and after in vitro degradation in PBS at 37° C. are analysed using a scanning electron microscope. Cross-sectioned samples are prepared using a razor blade for viewing their internal structure. The particles and their sectioned samples are mounted on metal stubs using double-sided adhesive tape and vacuum-coated with a platinum layer prior to the examination.

The particle samples are incubated in PBS (pH 7.4) at 37° C. The water uptake of the particles at predetermined time intervals is measured gravimetrically and calculated as the weight ratio of absorbed water to dried particles.

The in vitro coloring agent release analysis of the particles are carried out in triplicate at 37° C. in PBS (pH 7.4). A 40 mg amount of freeze-dried particles is dispersed in 10 ml PBS (pH 7.4) containing 0.1 (w/v) % Tween 80, which is agitated moderately. At predetermined time intervals, in vitro medium from each sample is removed and replaced with fresh PBS buffer. For the CA2-loaded particles, the CA2 content in the in vitro medium is directly analyzed using HPLC as stated above. For the CA1-loaded microspheres, an extraction method is employed to separate the water-insoluble coloring agent from the in vitro medium. Briefly, 10 ml hexane is added to the in vitro medium and the mixture is vigorously shaken for 5 minutes to extract CA1. The mixture is left to stand at room temperature overnight before the organic layer is drawn off and dried. A 5 ml volume of acetonitrile/water (85:15, v/v) is then added to dissolve the residues for further HPLC analysis. The CA1 standard samples are prepared according to the same procedures. However, for the CA1-CA2-loaded microspheres, after the extraction of CA1, the aqueous layer is collected to analyze CA2 content. The weight percentage of CA1 or CA2 cumulative release (%, w/w) is investigated as a function of incubation time.

### Example 3

### In Vitro Degradation of PLLA/P(CPP:SA) Particles

Particles are prepared by solvent evaporation. For the DW particles, seven batches are prepared as follows and pooled before sieving. Two solutions are prepared: 15% (w/v) PLLA in methylene chloride (4 ml) and 15% (w/v) P(CPP:SA) 20:80 in methylene chloride (4 ml). The two solutions are briefly mixed by gentle shaking and poured into 600 ml of 0.5% PVA in distilled water. Stirring is achieved by an overhead stirrer (Caframo, Type RZR50) at a rate of 450 rpm. As the solvent evaporates, the polymer phase separates and the PLLA phase engulfs the P(CPP:SA) 20:80 phase. Particles are stirred for 90-100 minutes before being collected by centrifugation, washed in distilled water, frozen, and lyophilized. They are sieved to size ranges of approximately 100 μm and stored at 20° C. Pooled particles are passed through a series of sieves and, subsequently, collected at each stage. Particles with diameters between 212 and 300 μm are used for the study.

SW PLLA particles are prepared in a similar manner, with eight batches being pooled. Particles are prepared from a 15% (w/v) solution in methylene chloride (8 ml) which is emulsified in 600 ml of 0.5% (w/v) PVA in distilled water by overhead stirring at a rate of 450 rpm. SW particles are stirred for 60-70 minutes before being processed as before. Particles of the same diameter (212-300 μm) as the DW particles are used for the study.

For the in vitro study, 50 mg aliquots of both the SW particles and the DW particles are suspended in 1 ml of phosphate buffered saline solution (PBS). After 1, 3 days, 1, 2 weeks, 1, 2, 4, and 6 months, an aliquot of each set of particles is washed with distilled water, frozen, and lyophilized for characterization by GPC, FTIR spectroscopy, DSC, and SEM. At each sampling time, the PBS solution is replaced with fresh PBS for the remaining samples.

Samples for SEM are freeze dried, mounted on metal stubs, and cross sectioned with a razor blade for viewing the internal structure. The samples are then sputter-coated with a 50-100 Å layer of gold-palladium (Polaron Instrument E5100) and viewed using a Hitachi S-2700 scanning electron microscope at an accelerating voltage of 10 kV.

Samples for transmission FTIR spectroscopy are prepared by casting dilute solutions (1% w/v in chloroform) of the samples onto sodium chloride (NaCl) crystals. All spectra are obtained using a Perkin-Elmer model 1725x spectrometer and manipulated using Infrared Data Manager software (Perkin-Elmer). Samples for DSC (5-15 mg) are sealed into aluminum sample pans (Perkin-Elmer Express). Thermal analyses of the particles are performed using a Model DSC 7 (Perkin-Elmer) equipped with controller model TAC 7/DX (Perkin-Elmer). After equilibration at 20° C. (1 min), samples are subjected first to heating from −20 to 200° C., cooled to −10° C., and finally reheated to 200° C., all at a rate of 10° C. min^{31 1}. Data from the first ramp are used in all cases. Thermograms are analyzed using Perkin-Elmer Thermal Analysis software for the calculation of glass transition temperatures (T_{g}), melting temperatures (Tₘ), and changes in enthalpy (ΔH).

The molecular weights of the polymers and the particles are estimated using a GPC system (Perkin-Elmer) consisting of a Isocratic LC pump model 250, LC column oven model 101, LC-30 RI detector, and 900 series interface. Samples are eluted in HPLC-grade chloroform (Fisher Scientific) through a PL gel 5μ mixed column and a 5 μm 50 Å⁻¹ column connected in series at a flow rate of 1.0 ml/min⁻¹ and a temperature of 40° C. The molecular weights of the polymers are determined relative to polystyrene standards (Polysciences, molecular weights between 1000 and 1,860,000 gmol⁻¹) using Turbochrom and TC*SEC software programs (Perkin-Elmer) for analysis. Samples are filtered before injecting to remove insoluble particulates when present.

### Example 4

### In Vivo Degradation of PLLA/P(CPP:SA) Particles

Aliquots of 30 mg of particles are loaded into glass vials and the vials are plugged with cotton and packaged for cold cycle ethylene oxide sterilization (EtO). Three aliquots of particles are prepared for each rat, two to be implanted intramuscularly in the quadriceps and one to be implanted subcutaneously between the shoulder blades. In order to provide enough material to be later extracted for characterization of the polymer, four rats are used for each timepoint. The timepoints for the study are 1 and, 2 weeks, 1, 2, 4, and 6 months. With four rats per timepoint and 6 timepoints, 24 rats are implanted with DW PLLA and P(CPP:SA) 20:80 microspheres. A second set of 24 rats are implanted with SW PLLA microspheres for comparison. Rats are anesthetized with a 60 mg kg⁻¹ IP injection of sodium pentobarbital (Nembutal®). The implant sites are shaved and swabbed first with alcohol and then by an iodinated solution.

Using sterile techniques, a 1 cm long skin incision is opened over the quadriceps. The incision is then continued into the muscle. The particles are then carefully poured into the muscle incision and the muscle fascia is closed with simple interrupted sutures of 5-0 Vicryl to secure the implant. The skin incision is closed with a running subcuticular suture, also with 5-0 Vicryl. After the particles are implanted into both the hind limbs, the rat is turned onto its stomach and a 1 cm incision is made through the skin between the shoulder blades. A small subcutaneous pocket is created and the particles are introduced into this site. The skin incision is closed with a running subcuticular stitch with 5-0 Vicryl. The rats are allowed to recover on a heating pad post-operatively. NIH guidelines for the care and use of laboratory animals (NIH publication #85-23 Rev. 1985) are observed. At the designated timepoints after implantation, rats are sacrificed by overdose with IP and intracardiac Nembutal® or CO₂ inhalation.

The implant sites are then explanted for analysis. One subcutaneous implant and one intramuscular implant from each group of rats are carefully excised along with the surrounding tissue for histological evaluation. These are placed in 4% (w/v) paraformaldehyde in PBS for 6-8 hours and then incubated overnight in 30% (w/v) sucrose in PBS. The fixed samples are mounted in embedding medium, frozen, and then sectioned on a cryostat into 40 μm thick sections for microscopy. The remaining explanted samples are pooled, frozen, and lyophilized in preparation for polymer extraction. The dried tissue is ground using a mortar and pestle and chloroform is added. The slurry is filtered through 0.2 μm PVDF syringe filters and the chloroform is allowed to evaporate from the filtrate. The dry, extracted polymer is then characterized by GPC, FTIR spectroscopy, and DSC. The original particles after fabrication as well as after ethylene oxide sterilization are characterized by the same methods.

1.-52. (canceled)

53. A semi-permanent tattoo composition comprising:
(i) a plurality of particles, wherein each particle comprises:
(a) a polymer that is bioabsorbable and biodegradable, wherein the polymer comprises poly D-lactic acid (PDLA), poly L-lactic acid (PLLA), poly(lactic-co-glycolic acid) (PLGA), polycaprolactone (PCL), or polyethylene glycol (PEG); and
(b) a coloring agent having a molecular weight of 5 to 10×10⁶ Daltons; and
(ii) a carrier solution,
wherein the coloring agent is incorporated in the polymer's matrix; and
wherein the particles are present in the carrier solution at a concentration of about 5% w/v to about 60% w/v of the carrier solution, wherein about is ±20%.

54. The composition of claim 53, wherein the composition is useful as a semi-permanent tattoo ink which retains its vibrancy for 2 months to 12 months.

55. The composition of claim 53, wherein the particles are present in the carrier solution at a concentration of about 340 mg/ml to about 600 mg/ml, wherein about is ±20%.

56. The composition of claim 53, wherein the particles are present in the carrier solution at a concentration of from about 340 mg/ml to about 370 mg/ml, from about 370 mg/ml to about 400 mg/ml, from about 400 mg/ml to about 430 mg/ml, from about 430 mg/ml to about 450 mg/ml, from about 450 mg/ml to about 480 mg/ml, from about 480 mg/ml to about 510mg/ml, about 510 mg/ml to about 540 mg/ml, about 540 mg/ml to about 570 mg/ml, or about 570 mg/ml to about 600 mg/ml, wherein about is ±20%.

57. The composition of claim 53, wherein the particles have an average diameter of less than 100 μm.

58. The composition of claim 53, further comprising a humectant.

59. The composition of claim 53, further comprising a buffer.

60. The composition of claims 53, further comprising a surfactant.

61. The composition of claim 53, wherein the coloring agent is a color additive subject to FDA color additive regulations.

62. The composition of claim 61, wherein the coloring agent is D&C Yellow 10, D&C Orange 4, D&C Red 33, D&C Red 22, D&C Yellow 8, D&C Green 5, D&C Red 17, D&C Red 21, D&C Red 27, D&C Yellow 11, D&C Violet 2, D&C Green 6, D&C Red 30, D&C Red 31, D&C Red 28, D&C Red 7, D&C Red 6, D&C Red 34, D&C Yellow 10, D&C Red 36, D&C Green 8, D&C Yellow 10 Lake, D&C Red 21 Lake, D&C Red 33 Lake, FD&C Red 40 Lake, D&C Red 27 Lake, D&C Red 28 Lake, D&C Red 30 Lake, D&C Red 36 Lake, D&C Red 6Lake, D&C Red 7 Lake, D&C Black 2, Ext D&C Violet 2, Ext D&C Yellow 7, FD&C Red 40, FD&C Yellow 5, FD&C Blue 1, FD&C Blue 2, FD&C Green 3, FD&C Red 4, FD&C Yellow 6, FD&C Red 3, FD&C Yellow 5 Lake, FD&C Yellow 6 Lake, or FD&C Blue 1 Lake.

63. The composition of claim 53, wherein the coloring agent is a dye, is a pigment, is fluorescent, or is phosphorescent.

64. The composition of claim 53, wherein the coloring agent is beta carotene or sodium copper chlorophyllin.

65. The composition of claim 53, wherein the polymer comprises PLGA.

66. The composition of claim 53, wherein the particles are present in the carrier solution in an amount that is effective to induce aggregation of the particles upon their incorporation into a subject's skin.

67. A method of cosmetically tattooing a subject, comprising intradermally administering to the subject a cosmetically effective amount of the composition of claim 53.

68. The method of claim 67, wherein the tattooing provides a tattoo that partially or fully changes its color as the particles degrade.
